(19) United States
(12) Patent Application Publication
(10) Pub. No.: US 2011/0027909 A1
(43) Pub. Date: Feb. 3, 2011

(54) Chimera Compositions and Methods of Use

(76) Inventors: Selena Bartlett, Berkeley, CA (US); Antonello Bonci, San Francisco, CA (US); Carolina Haass-Koffler, San Francisco, CA (US); Mohammed Naeemuddin, Fremont, CA (US)

Correspondence Address: Convergent Law Group LLP, P.O. BOX 1329, MOUNTAIN VIEW, CA, 94042, US

(21) Appl. No.: 12/754,499

(22) Filed: Apr. 5, 2010

Related U.S. Application Data
(60) Provisional application No. 61/166,632, filed on Apr. 3, 2009.
(60) Provisional application No. 61/182,032, filed on May 28, 2009.

(51) Int. Cl. G01N 33/53; C07K 14/00; C07K 2/00; C07K 16/00; C07H 3/00

(52) U.S. Cl. 436/501; 530/350; 530/300; 530/387.1; 536/1.11

(57) ABSTRACT

This invention is directed to novel compositions, process methods, research tools, and use of these in the identification and development of novel therapeutic and/or diagnostic products. The compositions of the invention are chimera proteins that in essence recreate and/or potentiate one or more protein complex interactions that occur in vivo in the modulation of biological processes.

### CROSS-REFERENCE TO RELATED APPLICATIONS

This application claims priority to provisional application Ser. No. 61/166,632 filed Apr. 3, 2009, and provisional application Ser. No. 61/182,032, which are both incorporated by reference in their entirety

### FIELD OF THE INVENTION

This invention relates to compositions, research tools, and methods of use for drug discovery. In particular, the invention relates to chimera proteins used to identify modulators of biological activity mediated through transmembrane proteins.

### BACKGROUND OF THE INVENTION

In the following discussion certain articles and methods will be described for background and introductory purposes. Nothing contained herein is to be construed as an “admission” of prior art. Applicant expressly reserves the right to demonstrate, where appropriate, that the articles and methods referenced herein do not constitute prior art under the applicable statutory provisions.

G protein-coupled receptors (GPCRs), also known as seven transmembrane domain receptors, 7TM receptors, heptahelical receptors, and G protein-linked receptors (GPLR), form the largest class of cell surface receptors in humans and one of the most important families of drug targets. They comprise a large protein family of transmembrane receptors involved in numerous signal transduction pathways and linked cellular responses. The ligands that bind and activate these receptors include light-sensitive compounds, odors, pheromones, hormones, and neurotransmitters, and vary in size from small molecules to peptides to large proteins.

GPCRs are prominent components of drug portfolios in small and large pharmaceutical companies alike, and many drug discovery firms focus exclusively on these receptors. Whereas, in other types of receptors that have been studied, ligands bind externally to the membrane, the ligands of GPCRs typically bind within the transmembrane domain, or as with the chemokines in a multisite binding manner with part of the chemokine binding to the N terminus and another part binding within the transmembrane portion. The activation of GPCRs also generally involves the formation of a complex of proteins rather than binding and activation by a single, specific ligand. Thus, a single GPCR can be involved in multiple processes, with the specificity conferred by the combination of molecules involved in the activation of the signaling pathway or process. This can present a particular challenge for targeting GPCRs for modulation of specific biological processes, as the specificity is generally conferred by a complex involving multiple protein:protein interactions. Targeting the molecule itself may have unintended effects on other processes, and result in toxicity due to the inadvertent targeting of multiple biological pathways.

Directed efforts to identify drugs that modify specific GPCR signaling pathway protein complexes have been limited in large part by an inability to recreate such complex protein interactions and perform measurements in an ex vivo setting. The present invention provides compositions, research tools, and assays that address this need.

### SUMMARY OF THE INVENTION

This Summary is provided to introduce a selection of concepts in a simplified form that are further described below in the Detailed Description. This Summary is not intended to identify key or essential features of the claimed subject matter, nor is it intended to be used to limit the scope of the claimed subject matter. Other features, details, utilities, and advantages of the claimed subject matter will be apparent from the following written Detailed Description, including those aspects illustrated in the accompanying drawings and defined in the appended claims.

This invention is directed to novel compositions, process methods, research tools, and use of these in the identification and development of novel therapeutic and/or diagnostic products. The compositions of the invention are chimera proteins that in essence recreate and/or potentiate one or more protein:protein interactions that occur in vivo in the modulation of biological processes.

The chimera compositions of the invention comprise 1) a peptide having an N-terminal extracellular domain from a GPCR, a transmembrane region from a GPCR, and an intracellular signaling domain from a GPCR and 2) a peptide corresponding to a protein that associates with a GPCR complex in the modulation of a biological process. The second peptide is fused to the first peptide, and preferably to the N-terminal extracellular domain of the first peptide, and allows functional expression of the chimera composition in cells, which allows the fused chimera to adopt the appropriate conformational configuration and to insert into the appropriate membrane in a manner that preserves the GPCR signaling activity of the first peptide. The chimera thus has preserved signaling activity in functional assays, including assays in mammalian cells, and is useful in the identification or investigation of GPCR activity.

In some aspects of the invention, the chimera compositions comprise a substantially complete amino acid sequence of a particular GPCR, and thus the first peptide comprises an N-terminal extracellular domain, transmembrane region, and an intracellular signaling domain corresponding to a single GPCR. The second peptide of the chimera fusion corresponds to a protein that associates with the particular GPCR in the modulation of a biological process.

In other aspects of the invention, the chimera compositions comprise the N-terminal extracellular domain and transmembrane region of a first GPCR, and the intracellular signaling domain of a second GPCR. This may be useful to identify binding partners that modulate the first GPCR via binding to epitopes on the N-terminus and/or transmembrane of the first GPCR, but using established assays with the ability to measure intracellular activity of the second GPCR. The second peptide of the chimera fusion corresponds to a protein that associates with the first GPCR in the modulation of a biological process.

In yet other aspects of the invention, the chimera compositions comprise the N-terminal extracellular domain of a first GPCR, and the transmembrane region and intracellular signaling domain of a second GPCR. This may be useful to identify binding partners that modulate the first GPCR via binding to epitopes on the N-terminus of the GPCR utilizing one or more ligands that are known to bind within the transmembrane region of the second GPCR, again using established assays with the ability to measure activity of the second GPCR. The transmembrane region can be selected based desired ligand binding to the GPCR that will be controlled in the functional assay, since the ligands of GPCRs often bind within the transmembrane domain of the protein.

In these aspects, the second peptide comprises all or a functional portion of a protein that binds to the relevant GPCR portion of the first peptide and/or facilitates association of the specific protein complex that modulates activity of the first peptide, either through direct binding to the first peptide or through binding of a protein complex partner that binds to both the first and second peptide of the chimera composition.

Both the first and the second peptide may include amino acid sequence variants that are naturally occurring (e.g., due to genetic polymorphisms within a population) or that are added to confer a desirable characteristic to the composition (e.g., mutations introduced to increase stability, to aid in production, and/or to aid in isolation of the composition).

The compositions of the invention may be created using recombinant technology, or they may be associated following expression of the proteins using synthetic or biological linkers. In a preferred aspect, the composition is produced as a single recombinant protein in a cell.

One significant use of the composition is as a research tool specifically for the discovery and development of therapeutic products for modulation of a biological process involved in a disease, disorder and/or physiological behaviors such as cognition or memory. The research tool may be useful in various aspects of drug discovery and investigation, including without limitation the initial identification of a drug candidate, the confirmation of activity of a drug candidate; and the identification of activity in an existing pharmaceutical product.

In another specific aspect, the invention acts as natural allosteric modulator by increasing GPCR responsiveness to its natural ligand. This assay facilitates the discovery of ligands and compounds that act as allosteric modulators of GPCR signaling assays.

Another use of the composition is as a research tool specifically used as a diagnostic tool to detect the presence or absence of molecules necessary for the modulation of a biological process involved in a disease or disorder.

Thus, in one aspect the invention includes research tools comprising the compositions of the invention, and uses of such research tools in identification, investigation and/or confirmation of activity of binding partners that are useful as therapeutic agents. The present invention thus encompasses binding partners that are isolated using the method of the invention and uses of such binding partners in either a therapeutic or a diagnostic setting.

In one specific aspect, the invention provides a research tool for the identification and/or confirmation of activity of an agent with binding to sites on one portion of the chimera, e.g., a binding partner that binds to one or more epitopes of a single peptide within the chimera (e.g., an epitope on the first GPCR peptide of the chimera). In another preferred aspect, the binding partner is capable of binding to sites on two distinct portions of the chimera proteins, e.g., binding to a first epitope on the GPCR peptide of the chimera and a second epitope on the second peptide.

In another aspect, the invention is directed to assays for identification of GPCR signaling activity that comprise the chimera proteins of the invention. Use of the research tools of the invention can in essence recreate one or more GPCR interactions that occur in vivo in the modulation of a biological process, thus potentiating selective binding of binding partners that require the association of two or more members of the GPCR signaling complex to modulate activity.

In a more specific aspect, the invention is directed to chimera proteins that in essence recreate one or more Class A GPCR interactions that occur in vivo in the modulation of a biological process. These compositions comprise 1) a first peptide corresponding to a Class A GPCR and 2) a second peptide that corresponds to a binding partner known to associate in a complex with the Class A GPCR in the modulation of a biological process. The first peptide may correspond to all or a relevant portion of the Class A GPCR involved in the target biological process. The second peptide comprises all or a relevant portion of a protein that binds to the first peptide and/or facilitates binding of another binding complex member peptide to the first Class A GPCR peptide in the modulation of a biological process.

In another more specific aspect, the invention is directed to chimera proteins that can recreate one or more Class B GPCR interactions that occur in vivo in the modulation of a biological process. These compositions comprise 1) a first peptide corresponding to a Class B GPCR and 2) a second peptide that corresponds to a binding partner known to associate in a complex with the Class B GPCR in the modulation of a biological process. The first peptide may correspond to all or a relevant portion of the Class B GPCR involved in the target biological process. The second peptide comprises all or a relevant portion of a protein that binds to the first peptide and/or facilitates binding of another binding complex member peptide to the first Class B GPCR peptide in the modulation of a biological process.

In yet another more specific aspect, the invention is directed to chimera proteins that can recreate one or more Class C GPCR interactions that occur in vivo in the modulation of a biological process. These compositions comprise 1) a first peptide corresponding to a Class C GPCR and 2) a second peptide that corresponds to a binding partner known to associate in a complex with the Class C GPCR in the modulation of a biological process. The first peptide may correspond to all or a relevant portion of the Class C GPCR involved in the target biological process. The second peptide comprises all or a relevant portion of a protein that binds to the first peptide and/or facilitates binding of another binding complex member peptide to the first Class C GPCR peptide in the modulation of a biological process.

In yet another aspect, the present invention provides assays that are research tools for identification of a drug candidate for treatment of a biological process involving signaling through a GPCR. These assays comprise providing the chimera compositions of the invention, testing one or more binding partners for modulation of the functional activity of the research tool composition, and isolating the binding partners that display the desired change in functional activity of the research tool composition. The binding partners that display the desired change in functional activity of the research tool composition become drug candidates for the condition involving signaling through the GPCR.

In the assays of the invention, the research tool compositions can comprise an intracellular signaling domain and/or a transmembrane domain that correspond to the same GPCR as the N-terminal extracellular domain, or the compositions may comprise sequences from two or more GPCRs. In specific aspects, the research tool composition of the assay corresponds to a substantially complete amino acid sequence of a GPCR.

These and other aspects and uses of the invention will be provided in the written description.

### BRIEF DESCRIPTION OF THE FIGURES

FIG. 1 illustrates the structural elements of the construct used to create an CRF-BP_CRFR2 chimera.

FIG. 2 illustrates the structural elements of the construct used to create an CRF-BP(10 Kd)_CRFR2 chimera.

FIG. 3 is a line graph illustrating the ability of the expressed CRF-BP(FL)_CRFR2 chimera proteins to activate intracellular calcium release via signaling through Gq.

FIG. 4 is a line graph showing inhibition of CRF-induced (1 μM) stimulation in HEK 293 cells expressing the (CRF-BP(FL)-CRFR₂) by the CRF fragment, CRF₆₋₃₃ (10 pM-100 μM).

FIG. 5 is a line graph comparing the ability of the expressed CRF-BP_CRFR2 chimera proteins to activate intracellular calcium release via signaling through Gq with the inability of the CRF fragment, CRF (6-33) (1 pM-10 μM) to stimulate such intracellular calcium release.

FIG. 6 is a line graph showing the ability of the expressed CRF-BP(10 Kd)_CRF-R2 chimera proteins to activate intracellular calcium release via signaling through Gq.

FIG. 7 is a line graph showing the inability of untransfected HEK 293 cells or HEK 293 cells expressing the dopamine receptor to activate intracellular calcium release via signaling through Gq.

FIG. 8 illustrates the structural elements of the construct used to create a CRF-BP(FL)_NK₁R chimera.

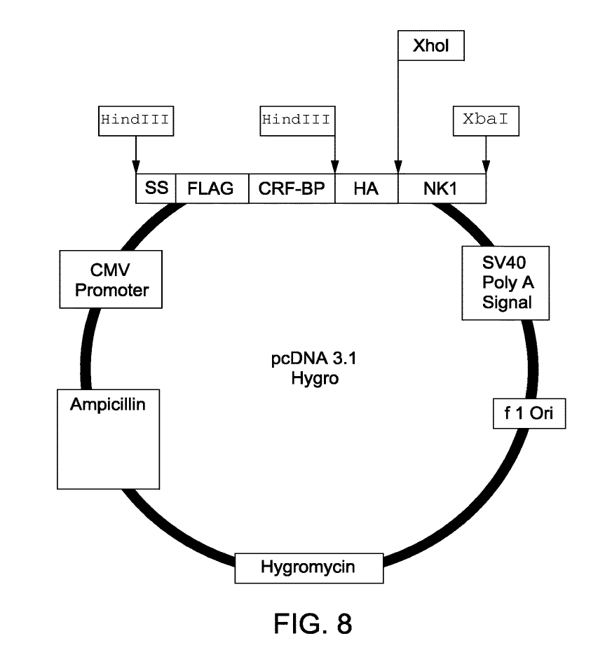

FIG. 9 is a line graph showing the ability of the expressed CRF-BP_{(FL)}_{_{—}}NK₁R chimera proteins to activate intracellular calcium release via signaling through Gq.

FIG. 10 is a line graph showing the ability of the expressed CRF-BP(FL)_NK₁R chimera proteins to activate intracellular calcium release via signaling through Gq.

FIG. 11 is a line graph showing the ability of NK₁R to activate intracellular calcium release via signaling through Gq.

FIG. 12 illustrates the structural elements of the construct used to create an IGF-BP2_CRFR2 chimera.

FIG. 13 is a line graph showing the ability of the expressed IGFBP2_CRFR2 chimera proteins to activate intracellular calcium release via signaling through Gq.

FIG. 14 illustrates the structural elements of the construct used to create an EGFR_CRFR2 chimera.

### DETAILED DESCRIPTION OF THE INVENTION

The practice of the techniques described herein may employ, unless otherwise indicated, conventional techniques and descriptions of organic chemistry, polymer technology, molecular biology (including recombinant techniques), cell biology, biochemistry, and sequencing technology, which are within the skill of those who practice in the art. Such conventional techniques include polymer array synthesis, hybridization and ligation of polynucleotides, and detection of hybridization using a label. Specific illustrations of suitable techniques can be had by reference to the examples herein. However, other equivalent conventional procedures can, of course, also be used. Such conventional techniques and descriptions can be found in standard laboratory manuals such as Green, et al., Eds. (1999), Genome Analysis: A Laboratory Manual Series (Vols. I-IV); Weiner, Gabriel, Stephens, Eds. (2007), Genetic Variation: A Laboratory Manual; Dieffenbach, Dveksler, Eds. (2003), PCR Primer: A Laboratory Manual; Bowtell and Sambrook (2003), DNA Microarrays: A Molecular Cloning Manual; Mount (2004), Bioinformatics: Sequence and Genome Analysis; Sambrook and Russell (2006), Condensed Protocols from Molecular Cloning: A Laboratory Manual; and Sambrook and Russell (2002), Molecular Cloning: A Laboratory Manual (all from Cold Spring Harbor Laboratory Press); Stryer, L. (1995) Biochemistry (4th Ed.) W.H. Freeman, New York N.Y.; Gait, “Oligonucleotide Synthesis: A Practical Approach” 1984, IRL Press, London; Nelson and Cox (2000), Lehninger, Principles of Biochemistry 3^{rd} Ed., W. H. Freeman Pub., New York, N.Y.; and Berg et al. (2002) Biochemistry, 5^{th} Ed., W.H. Freeman Pub., New York, N.Y., all of which are herein incorporated in their entirety by reference for all purposes. Before the present compositions, research tools and methods are described, it is to be understood that this invention is not limited to the particular methods, compositions, targets and uses described, as such may, of course, vary. It is also to be understood that the terminology used herein is for the purpose of describing particular embodiments only, and is not intended to limit the scope of the present invention which will be limited only by appended claims.

It must be noted that as used herein and in the appended claims, the singular forms “a,” “and,” and “the” include plural referents unless the context clearly dictates otherwise. Thus, for example, reference to “a composition” refers to one or mixtures of such compositions, and reference to “an assay” includes reference to equivalent steps and methods known to those skilled in the art, and so forth.

Unless defined otherwise, all technical and scientific terms used herein have the same meaning as commonly understood by one of ordinary skill in the art to which this invention belongs. All publications mentioned herein are incorporated herein by reference for the purpose of describing and disclosing devices, formulations and methodologies which are described in the publication and which might be used in connection with the presently described invention.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes both of the limits, ranges excluding either of those included limits are also included in the invention.

In the following description, numerous specific details are set forth to provide a more thorough understanding of the present invention. However, it will be apparent to one of skill in the art upon reading the specification that the present invention may be practiced without one or more of these specific details. In other instances, well-known features and procedures well known to those skilled in the art have not been described in order to avoid obscuring the invention.

### Definitions

Unless expressly stated, the terms used herein are intended to have the plain and ordinary meaning as understood by those of ordinary skill in the art. The following definitions are intended to aid the reader in understanding the present invention, but are not intended to vary or otherwise limit the meaning of such terms unless specifically indicated.

The term “allosteric modulator” is used to describe binding sites in the chimera compositions outside the conventional orthosteric protein binding site. Modulation of receptor signaling at such binding sites may affect receptor signaling without necessarily resulting in complete inhibition.

The term “antibody” is intended to include any polypeptide chain-containing molecular structure with a specific shape that fits to and recognizes an epitope, where one or more non-covalent binding interactions stabilize the complex between the molecular structure and the epitope. As antibodies can be modified in a number of ways, the term “antibody” should be construed as covering any specific binding member or substance having a binding domain with the required specificity. Thus, this term covers antibody fragments, derivatives, functional equivalents and homologues of antibodies, including any polypeptide comprising an immunoglobulin binding domain, whether natural or wholly or partially synthetic. Where bispecific antibodies are to be used, these may be conventional bispecific antibodies, which can be manufactured in a variety of ways (Holliger and Winter, 1993), e.g., prepared chemically or from hybrid hybridomas, or may be any of the bispecific antibody fragments mentioned above. It may be preferable to use scFv dimers or diabodies rather than whole antibodies. Diabodies and scFv can be constructed without an Fc region, using only variable domains, potentially reducing the effects of anti-idiotypic reaction. Other forms of bispecific antibodies include the single chain “Janusins” described in Traunecker et al, (1991). Such antibodies also include CRAbs, which are chelating antibodies which provide high affinity binding to an antigen, D. Neri, et al. J. Mol. Biol, 246, 367-373, and dual-variable domain antibodies as described in Wu C et al., Nat Biotechnol. 2007 November; 25(11):1290-7. Epub 2007 Oct. 14.

A “binding partner” is any molecule that is complementary to one or more regions on a chimera composition of the invention via association by chemical or physical means. For the purposes of the present invention, the binding partner may be a compound that facilitates binding of the composition with other members of a protein signaling complex, or a compound that interferes with the association of a known binding pair. Examples of binding partners that can be investigated and/or identified using this invention include, but are not restricted to: peptides, proteins (including derivatized or labeled proteins); antibodies or fragments thereof; small molecules; aptamers; carbohydrates and/or other non-protein binding moieties; derivatives and fragments of a naturally-occurring binding partners; peptidomimetics; and pharmacophores.

The term “biological process” as used herein includes both normal physiological processes, such as cognition, memory, neuroprotection, etc. as well as pathological processes, e.g. those involved in diseases and conditions such as depression, addiction, defective apoptotic activity, and the like.

The term “complementary” refers to the topological compatibility or interactive structure of interacting surfaces of a composition of the invention and a binding partner. Thus, the composition of the invention and its identified binding partners can be described as complementary, and furthermore, the contact surface characteristics are each complementary to each other. Preferred complementary structures have binding affinity for each other and the greater the degree of complementarity the structures have for each other the greater the binding affinity between the structures.

The term “CRF” refers to Corticotropin Releasing Factor, (also called Corticotropin-releasing hormone (CRH)), and includes any active fragments, modified peptides, derivatives or peptidomimetics that are based on corticotrophin releasing factor with substantially the same activity.

The term “CRF-BP” refers to one or more binding proteins that specifically bind to CRF and facilitate activity through either of its receptors, CRFR1 or CRFR2.

The term “diagnostic tool” as used herein refers to any composition or assay of the invention used in order to carry out a diagnostic test or assay on a patient sample. As a diagnostic tool, the composition of the invention may be considered an analyte specific reagent, and as such may form part of a diagnostic test regulated by a federal or state agency. The use of the compositions of the invention as a diagnostic tool is not intended to be related to any use of the composition in the development of therapeutic agents.

The term “epitope” refers to the portion of the composition of the invention which is delineated by the area of interaction with a binding partner.

The term “fused” when referring to a chimera of the invention refers to any mechanistic, chemical, or recombinant mechanism for attaching a specific member of a GPCR signaling complex to a GPCR or an active fragment thereof. The fusion of the second peptide to the first peptide may be a direct fusion of the sequences, with the second peptide directly adjacent to the first peptide, or it may be an indirect fusion, e.g., with intervening amino acid sequences such as an identifier or epitope tag sequence, a domain, a functional peptide or a larger protein. In certain aspects, the two peptides may be fused following co-expression in the cell, using high affinity binding sequences between the two peptides, such as biotin and avidin or strepavidin. In yet other examples, the two peptides are fused following expression of the GPCR in the cell and synthetic tethering of the second peptide to the N-terminus of the first GPCR peptide.

The terms “peptide,” “polypeptide,” and “protein” are used interchangeably herein, and refer to a polymeric form of amino acids of any length, which can include coded and non-coded amino acids, chemically or biochemically modified or derivatized amino acids, and polypeptides having modified peptide backbones.

The term “peptidomimetic” as used herein refers to a protein-like chain designed to mimic a peptide. They typically arise from modification of an existing peptide in order to alter the molecule's properties. For example, they may arise from modifications to change a molecule's stability, biological activity, or bioavailability.

The term “pharmacophore” is used herein in an unconventional manner. Although the term conventionally means a geometric and/or chemical description of a class or collection of compounds, as used here the term means a compound that has a specific biochemical activity or binding property conferred by the 3-dimensional physical shape of the compound and the electrochemical properties of the atoms making up the compound. Thus, as used here the term “pharmacophore” is a compound and not a description of a collection of compounds which have defined characteristics. Specifically, a “pharmacophore” is a compound with those characteristics.

The term “research tool” as used herein refers to any composition or assay of the invention used for scientific enquiry, academic or commercial in nature, including the development of pharmaceutical and/or biological therapeutics. The research tools of the invention are not intended to be therapeutic or to be subject to regulatory approval; rather, the research tools of the invention are intended to facilitate research and aid in such development activities, including any activities performed with the intention to produce information to support a regulatory submission.

The term “small molecule” refers to a molecule of a size comparable to those organic molecules generally used in pharmaceuticals. The term excludes biological macromolecules (e.g., proteins, nucleic acids, etc.). Preferred small organic molecules range in size up to about 5000 Da, more preferably up to 2000 Da, and most preferably up to about 1000 Da.

The term “selectively binds”, “selective binding” and the like as used herein, when referring to a binding partner (e.g., protein, nucleic acid, antibody, etc.), refers to a binding reaction which is determinative of the presence composition in heterogeneous population of molecules (e.g., proteins and other biologics). Thus, under designated assay conditions, the binding partner will bind to a composition of the invention at least two times the background and will not substantially bind in a significant amount to other proteins present in the sample. Typically, specific binding will be at least twice background signal or noise and more typically more than 10 to 100 times background. Thus, under designated conditions the binding partner binds to its particular “target” molecule and does not bind in a significant amount to other molecules present in the sample. A “target protein” as used herein includes any GPCR, including a portion or portions of a GPCR, that comprises one or more epitopes to which a binding partner selectively binds.

As used herein, the terms “treat,” “treatment,” “treating,” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse affect attributable to the disease. “Treatment,” as used herein, covers any treatment of a disease in a mammal, particularly in a human, and includes: (a) preventing the disease from occurring in a subject which may be predisposed to the disease but has not yet been diagnosed as having it; (b) inhibiting the disease, i.e., arresting its development; and (c) relieving the disease, e.g., causing regression of the disease, e.g., to completely or partially remove symptoms of the disease.

### The Invention in General

The present invention is based on the use of novel chimera protein-based compositions that in essence recreate the interaction of a GPCR and one or more of the proteins that normally interact with GPCRs in a signaling complex. The compositions of the invention can re-create functional activity of a GPCR in a cellular setting, and potentiate modulation of the GPCR by providing a GPCR and at least one other member of the GPCR signaling complex to the correct location for formation of the complex that modulates a biological process in a signaling-dependent manner. Use of the compositions of the invention as research tools provides high-throughput cell-based screening assays to identify molecules that interact with GPCRs based in part on their known naturally-occurring partners.

By providing a molecule with an inherent interaction between the receptor and at least one member of the GPCR signaling complex, identification of other members of the complex and/or binding partners that can somehow modify signaling through the receptor complex is greatly enhanced. The conservation of structure amongst the GPCR classes allows the invention to encompass chimeras having a large binding complex member fused to the N-terminus of virtually any GPCR.

Due to the large size and the conformational constraints of many of the naturally occurring GPCR signaling partners, it was a surprising result that mammalian cells would not only produce such fused chimera compositions, but that the fused composition would be inserted into the appropriate membrane and function appropriately in a cellular environment. The invention described herein sets forth a more general approach to creating novel compositions and assays for understanding GPCR signaling, an approach which takes advantage of the similarity in structure of this class of receptors and the ability of cells to somehow appropriately insert these chimera receptors across membranes despite the presence of a large peptide at the N-terminus of the receptor.

Due to the difficulties in recreating multiprotein complex interactions in an assay setting, e.g., to directly identify molecules that disrupt the interaction between GPCRs, their binding proteins and their ligands, the use of chimera compositions that in effect recreate at least one binding interaction of a complex removes one level of variability in creating the complex in vivo, and enhances the ability to identify binding partners that interact with one or both of these components in the signaling process. Thus, this invention overcomes the inherent difficulty in identifying molecules that disrupt the interaction of GPCRs with their binding partners. These chimera proteins can be used in in vitro assays or in ex vivo assays, as these proteins can be produced in stable cell lines and/or isolated as membrane fragments.

The chimera compositions of the invention are especially useful as research tools to identify binding partners that enhance signaling through GPCR complexes, or to identify binding partners that inhibit the appropriate proteins complex interactions necessary for signaling through a particular GPCR. Assays utilizing the chimera compositions of the invention allow testing of not just binding to GPCRs and/or other proteins in signaling complexes, but to also identify the effect binding partners have on functional cellular activity resulting from signaling through GPCRs. The ability to identify binding partners that display the desired change in functional activity is a great advantage of the invention, and will accelerated the identification and development of drug candidates having the desired changes in such cellular processes.

The functional change that is desirable in the treatment of the biological process will depend upon the desired increase or decrease of the GPCR signaling. Thus, the assay can be used to identify different effects of the functional activity of the GPCR, and may be used to identify drug candidates that are antagonists, partial agonists and/or agonists of the GPCR according to the need presented by the particular biological process to be treated.

### Exemplary GPCRs for Use in the Compositions of the Invention

G-protein-coupled receptors are a pharmacologically important protein family with approximately 450 genes identified to date. Pathways involving these receptors are the targets of hundreds of drugs, including antihistamines, neuroleptics, antidepressants, and antihypertensives. The GPCRs consist of seven transmembrane domains that are connected through loops. The N termini of these proteins are located extracellularly and C terminal is extended into the cytoplasmic space. Due to this topology, they are able to transduce the external signal into the cell.

GPCRs are classified into five major classes, which are further classified to subfamilies, each of which can be used in the creation and use of the compositions of the invention. The GPCR classes found to have activity in mammals include: Class A, the rhodopsin-like receptors, which is further divided into 19 subgroups (A1-A19); Class B, the secretin receptor family; Class C, the metabotropic glutamate/pheromone receptors; ocular albinism proteins (e.g., GPR143); and Class F, the frizzled/smoothened family, so named because of their initial discovery in Drosophila Melanogaster. A number of GPCRs are still considered “orphan receptors”, in that they act as receptors for stimuli that have yet to be identified. Any of these can be used in the creation and use of compositions of the invention.

The GPCR portion of the chimera composition can thus include sequences from: receptors for sensory signal mediators (e.g., light and olfactory stimulatory molecules); adenosine, bombesin, bradykinin, endothelin, γ-aminobutyric acid (GABA), hepatocyte growth factor, melanocortins, neuropeptide Y, opioid peptides, opsins, somatostatin, tachykinins, vasoactive intestinal polypeptide family, and vasopressin; biogenic amines (e.g., dopamine, epinephrine, norepinephrine, histamine, glutamate (metabotropic effect), glucagon, acetylcholine (muscarinic effect), and serotonin); chemokines; lipid mediators of inflammation (e.g., pro staglandins, pro stanoids, platelet-activating factor, and leukotrienes); and peptide hormones (e.g., calcitonin, C5a anaphylatoxin, follicle-stimulating hormone (FSH), gonadotropic-releasing hormone (GnRH), neurokinin, thyrotropin-releasing hormone (TRH), and oxytocin).

In one specific aspect, the GPCR portion of the chimera corresponds to receptors involved in signaling in the central nervous system and anterior pituitary, as exemplified by the Class B GPCRs, CRFR1 and CRFR2. These receptors are believed to play a central role in depression, anxiety, and stress disorders. CRFR1 mediates anxiety and depression behaviors and HPA axis stress response, and may be involved in the initiation of escapable and controllable stressors. CRFR2, on the other hand, is known to play a role in such responses, either to reinstate homeostasis to counteract CRFR1 activity or to mediate anxiety and depression responses caused by inescapable stressors. Hauger R L et al., CNS Neurol Disord Drug Targets 2006 August 5:453-479. The ability to identify molecules that selectively modulate signaling of one or both of these receptors could be instrumental in not only understanding these pathways, but also in identification and development of therapeutics useful for control of such neurological responses.

In another specific aspect, the GPCR portion of the chimera composition corresponds to a chemokine receptor. Chemokines and their receptors play a pivotal role in lymphocyte trafficking, recruiting and recirculation. Chemokine receptors are GPCRs belonging to the rhodopsin superfamily. They have an N-terminus outside the cell, three extracellular domains, three intracellular loops and a C-terminus in the cytoplasm which contains serine and threonine phosphorylation sides. Unusually for GPCRs, nearly all chemokine receptors have multiple high-affinity ligands for a single receptor. CCR5, for example, binds CCL3 and CCL4 as well as CCL5. Binding of a chemokine to its specific receptor on the cell surface results in chemotaxis towards the source of the chemokine. Based on their ligand specificity, chemokine receptors can be divided into two major groups, CXCR and CCR, based on the two major classes of chemokines. Thus far six CXC receptors, one CX3C and twelve CC receptors have been identified. The creation of chimera compositions may help to tease out the specific interactions necessary for signaling through these receptors, and again aid in identification of binding partners with potential therapeutic effects.

The GPCR sequences of the compositions of the invention can also be modified for numerous reasons, including to enhance their usefulness in assays or in vivo stability; to include identifying sequences, such as epitope tags (HA tags, FLAG tags, etc.) or fluorescent indicator proteins; or to provide sequences that aid in isolation of the compositions, etc. Examples of modifications that can be used in the compositions of the invention include those described in US20080009551, in which a GPCR is modified to produce a ligand upregulatable GPCR; luciferase tagged mutants as described in Ramsay et al., Br J Pharmacol. 2001 133:315-23 and McLean et al., Mol Pharmacol. 1999 56:1182-91; constitutively active receptors, as described in McLean et al., Mol Pharmacol. 2002 62:747-55, Samama et al. J Biol Chem. 1993 268:4625-36, Parnot et al., Trends Endocrinol Metab. 2002 13:336-43, and Teitler et al, Curr Top Med Chem. 2002 2:529-38; sequences to facilitate domain swapping for chimera production; modifications to allow interaction and/or use of specific assay components, e.g., sequences to facilitate the use of the PathHunter™ β-Arrestin assays; C-terminal identifiers such as fluorescent proteins (e.g., green fluorescent protein, yellow fluorescent protein, etc.); receptor modifications (e.g., channel rhodopsin modifications) to allow optogenetic applications and detection of light-based activation; and biarsenical labeling reagents such as TC-FlAsH and TC-ReAsH (Invitrogen, Carlsbad, Calif.) which allow drug-based protein detection (see also Adams S R et al., J Am Chem Soc. 2002 May 29; 124(21):6063-76); and the like.

In one specific aspect, the compositions of the invention comprise GPCR sequences that have been specifically engineered to more finely control activation of the receptors. For example, certain engineered GPCRs, called receptors activated solely by synthetic ligands (RASSLs), are unresponsive to endogenous ligands but can be activated by nanomolar concentrations of pharmacologically inert, drug-like small molecules. This allows precise spatiotemporal control of GPCR signaling in vivo, and thus may be very useful in constructing compositions for identification of biological modulators. Currently, RASSLs exist for the three major GPCR signaling pathways (G(s), G(i) and G(q)). See Conklin B R et al., Nat Methods. 2008 August; 5(8):673-8. The invention thus includes these and other similarly-modified receptors for use in the compositions, research tools and assays.

Design of Compositions and Assays Based on Polymorphism and/or Tissue Specific Expression of GPCRs

Responses to currently available drugs targeted to GPCRs can show substantial variability between subjects, and attempts to use clinical factors as a means to predict individual drug responses have had limited success. One key challenge for design of biologics with desired pharmaceutic properties, including a desired safety profile, is the ability to identify drug candidates that will selectively act on a particular receptor in a target tissue and largely spare those receptors in other tissues, thereby minimizing potential adverse effects. As GPCR polymorphisms may have tissue-, cell type- or ligand-specific effects on protein production and drug responses, it may be desirable to design specific compositions for targeting GPCRs based on differences in known alleles with specific polymorphisms, as factors intrinsic to the biochemical properties of the different receptors may contribute to such heterogeneity and may be linked to disease susceptibility and/or efficacy and toxicity of therapeutic agents in certain patient populations.

Consequently, specific compositions of the invention may be used to identify drug candidates that are tailored to specific patient populations to reflect the polymorphic nature of the GPCR coding regions within these populations. Compositions that contain certain amino acids may be used to identify binding partners that specifically modulate signaling through such population-specific GPCRs. Multiple compositions of the invention comprising variant sequences corresponding to a single GPCR can be used to reflect structural variability between patient groups.

Alternatively, design of the chimera compositions may aid in identifying drug candidates appropriate for a larger patient population. Identification of drug candidates that will bind and have clinical effect across patient groups can be facilitated by identifying binding partners that selectively bind to portions of a GPCR that do not include such structural variations, thus ensuring that the maximum number of patients will benefit.

GPCR polymorphisms can not only produce proteins with tissue-specificity but can also those that act in a ligand-specific manner, termed “ligand-directed signaling”, whereby activation of a given GPCR by two chemically distinct ligands leads to differential signaling responses Pauwels P J et al., Journal of Pharmacology and Experimental Therapeutics. 2003; 305:1015. In certain aspects of the invention, it may be desirable to design chimera compositions that better address the potential effects of these polymorphic changes in subsets of the population. Examples of such polymorphisms in GPCR alleles include, but are not limited to, the following:

| | Drugs and some key | | |
|---|---|---|---|
| Receptor | indications | Polymorphisms | Relevance |
| α_{1A}-adrenergic | Antagonists (e.g. | C1475T | Short- and long-term |
| receptor | tamsulosin) to treat | | antagonist effects |
| | micturition (bladder | | apparently not affected |
| | emptying) disorders | | |
| | associated with enlarged | | |
| | prostate glands | | |
| β₁-adrenergic | Antagonists (e.g. | Ser49Gly | Arg389 linked to |
| receptor | propranolol, atenolol, | Arg389Gly | increased antagonist |
| | metoprolol, carvedilol) to | | effect |
| | treat essential | | |
| | hypertension or | | |
| | congestive heart failure | | |
| β₂-adrenergic | Agonists (e.g. | Arg16Gly | Possibly reduced |
| receptor | terbutaline, salbutamol, | Gln27Glu | responses with Ile164 |
| | formoterol, salmeterol) | Thr164Ile | otherwise no consistent |
| | for treatment of | | association with drug |
| | obstructive airway | | responsiveness |
| | disease or premature | | |
| | labor | | |
| D₂ dopamine | Antagonists (e.g. | −141C Ins/Del | Reduced antagonist |
| receptor | haloperidol) to treat | Taq1A | response with Del or |
| | schizophrenia | | homozygous A2 allele |
| | Agonists (e.g. levodopa) | | No consistent associations |
| | for the treatment of | | with therapeutic response |
| | Parkinson's disease | | or side effects of agonists |
| D₃ dopamine | Antagonists (e.g. | Ser9Gly | Increased risk of tardive |
| receptor | haloperidol) in the | | dyskinesia with Gly allele |
| | treatment of | | |
| | schizophrenia | | |
| 5-HT_{2A} receptor | Antagonists (e.g. | T102C | Reduced response to |
| | clozapine) to treat | | clozapine with C allele |
| | schizophrenia | | Possibly reduced response |
| | Indirect agonists (e.g. | | to agonists with |
| | fluvoxamine) for the | | homozygous T allele |
| | treatment of depression | | |
| 5-HT_{2C} receptor | Antagonists (e.g. | Multiple | Genotypes associate with |
| | clozapine) to treat | polymorphisms in | therapeutic response and |
| | schizophrenia | promoter and coding | with side effects such as |
| | | region in linkage | tardive dyskinesia and |
| | | disequilibrium | weight gain |

Source: Insel, PA et al., Biochim Biophys Acta. 2007 April; 1768(4): 994-1005.

The chimera compositions can be constructed to include GPCR sequences having these polymorphic changes, and thus potentially address issues of non-responsiveness of certain patient populations to currently available therapies. In addition, use of these compositions as research tools in assays can address issues of safety for patients that have displayed a serious adverse reaction due to a protein encoded by a particular polymorphic GPCR allele.

### Binding Affinities

The strength of the interaction of a binding partner with a composition can be characterized by its “binding affinity” to a given binding site or epitope. In the field of immunology, antibodies are characterized by their “binding affinity” to a given binding site or epitope. Every antibody is comprised of a particular 3-dimensional structure of amino acids, which binds to another structure referred to as an epitope or antigen.

The selective binding of a binding partner to a composition is a simple bimolecular, reversible reaction, not unlike the binding of an antibody to its antigen. For example, if the antibody is represented by Ab and the antigen by Ag, the reaction can be analyzed by standard kinetic theory. Assuming a single binding site the reaction is represented by the equation I as follows:

$$Ag + Ab {{⇌}^{{k}_{1}}}_{{k}_{2}} Ag - Ab \qquad I$$

where Ag-Ab is the bound complex. The forward and reverse binding reactions are represented by rate constants k₁ and k₂ respectively. The “binding affinity” of the antibody to the antigen is measured by the ratio of complexed to free reactants at equilibrium. The lower the concentration of the reactants at equilibrium, the higher the binding affinity of the antibody for the antigen. In the field of immunology, the binding affinity is represented by an “affinity constant” which is represented by the symbol “K” or sometimes referred to as “Kₐ”. The “K” is defined by the equation II as follows:

$$K = \frac{[ Ag - Ab ]}{[ Ag ] [ Ab ]} = \frac{{k}_{1}}{{k}_{2}} \qquad II$$

where the brackets denote concentration in moles per liter or liters per mole.

A typical value for the binding affinity Kₐ which is also referred to as “K” and is the “affinity constant” which for a typical antibody is in a range of from about 10⁵ to about 10¹¹ liters per mole. The Kₐ is the concentration of free antigen needed to fill half the binding sites of the antibody present in solution with the antigen. If measured in liters per mole a higher Kₐ (e.g. 10¹¹) or higher affinity constant indicates a large volume of solvent, a very dilute concentration of free antigen, and as such indicates the antibody has a high binding affinity for the epitope.

If the Kₐ is measured in moles per liter a low Kₐ (e.g. 10⁻¹¹) indicates a less concentrated solution of the free antigen needed to occupy half of the antibody binding sites, and as such a high binding affinity.

Equilibrium is achieved in order to measure the Kₐ. More specifically, the Kₐ is measured when the concentration of antibody bound to antigen [Ag-Ab] is equal to the concentration of the antibody [Ab]. Thus, [Ag-Ab] divided by [Ab] is equal to one. Knowing this, the equation II above can be resolved to the equation III as follows:

$$K = \frac{1}{[ Ag ]} \qquad III$$

In equation III the units for K are liters per mole. Typical values in liters per mole are in a range of from about 10⁵ to about 10¹¹ liters per mole.

The inverse of the above equation is K=[Ag] where the units for K are in moles per liter, and the typical values are in a range of 10⁻⁶ to 10⁻¹² moles per liter.

The above shows that typical binding affinities can vary over six orders of magnitude. Thus, what might be considered a useful antibody might have 100,000 times greater binding affinity as compared to the binding affinity of what might be considered a different antibody, which is also considered useful.

Based on the above it will be understood that binding characteristics of an antibody to an antigen can be defined using terminology and methods well defined in the field of immunology. So, too, can the binding characteristics of a ligand to its target can be defined. The binding affinity or “K” of a ligand can be precisely determined.

Those skilled in the art will understand that a high degree of binding affinity does not necessarily translate to a highly effective drug. Thus, when obtaining binding targets that are drug candidates, candidates showing a wide range of binding affinities may be tested to determine if they obtain the desired biochemical/physiological response. Although binding affinity is important, some drug candidates with high binding affinity are not effective drugs and some drug candidates with low binding affinity are effective drugs. The functional assessment of any binding partners identified or investigated using the compositions of the invention is thus a critical part of any drug design to ensure the drug candidate meets the desired specifications.

### Functional Assays

The chimera compositions of the invention are useful as either research or diagnostic tools in functional assays, including: assays used to understand physiological processes; assays to identify new binding partners (including drug candidates) that selectively bind to GPCRs and/or proteins in GPCR signaling complexes and modulate specific signaling processes; and assays to test known compounds (including synthetic, recombinant or naturally-occurring compounds) for their effect on signaling through GPCRs, and the like. It is known in the pharmaceutical arts that binding affinity to a target and efficacy do not necessarily correlate, and that identification of functional changes conferred by a binding partner is a much better predictor of efficacy than binding affinity alone. The chimera compositions of the invention are especially powerful in identification of binding partners with functional activity rather than just affinity, as the chimeras not only can recreate functional activity of GPCRs but also provide potentiation of the signaling pathway through pre-existing interaction of the GPCR and at least one binding partner.

Functional assays for use with the compositions of the present invention include biochemical assays which can be correlated with in vivo efficacy for a physiological process, ex vivo cell-based assays for measurement of a physiological process, in vivo assays for direct or indirect measurement of a physiological process, etc.

The functional assays of the invention are any assays that correlate with in vivo modulation of a process. Examples of cell-based assays for use with the present invention include, but are not limited to, high throughput binding screening; assays to measure cell proliferation, death necrosis and/or apoptosis; flow cytometry assays; metabolic assays measuring labeling or turnover; phase and fluorescence microscopy; receptor phosphorylation and/or turnover; cell signaling assays; immunohistochemistry studies; reporter gene assays, and subcellular fractionation and localization. More specific examples of such assays are: FLIPR to detect changes in intracellular calcium concentration; CACO to predict human oral absorption of drug compounds; and cell-based ELISA assays to detect and quantify cellular proteins including post-translational modifications associated with cell activation; [³⁵GTPγS] binding assays, PathHunter™ beta-arrestin technology, SureFire™ MAPkinase assays; PathHunter™ MAP kinase assays; and radioligand binding assays.

Biochemical assays can also be used to correlate binding with efficacy in the methods of the invention. These include, but are not limited to, spectrophotometric assays, fluorometric assays, calorimetric assays, chemiluminescent assays, radiometric assays, chromatographic assays, colorimetric assays, and substrate specificity inhibitor kinase assays. Specific examples are: luciferase assays, in which firefly luciferase protein catalyzes luciferin oxidation and light is generated in the reaction, and which is frequently used as a report gene for measuring promoter activity or transfection efficiency; electrophoresis; gas-liquid chromatography; Förster resonance energy transfer (FRET); and use and detection of activation by RASSLs.

In a specific aspect, in vivo assays are utilized to provide a correlation of binding affinity with efficacy in modulating a target. Examples of in vivo functional assays are radiolabelling assays, fluorescent protein expression assays, in vivo capture assays, NMR spectroscopy, or assays specifically designed to identify efficacy in an animal model of a pathological process. For example, in treatment of certain diseases or disorders, such as infectious diseases, therapeutics need to be initially tested in in vivo models due to the complex physiological parameters involved with efficacy.

### Use of Research Tools to Indentify Agonists and Antagonists

Drugs targeting GPCRs generally have fallen into two categories: agonists, which are drugs that mimic the actions of endogenous transmitters and hormones to stimulate GPCRs, and antagonists, which have no intrinsic activity of their own but which block activation of the GPCRs by agonists.

For example, agonists can be distinguished as full agonists, partial agonists, and inverse agonists, each with its own sets of advantage and disadvantages as therapeutics. A full agonist is a drug that produces the same maximal effect as the endogenous neurotransmitter or hormone. Partial agonists are drugs that bind to GPCRs in a manner that produces less of an effect than full agonists. Partial agonists can antagonize full agonists. As a consequence, partial agonists exhibit duality in that they bind to GPCRs in a manner similar to both an agonist and an antagonist.

Partial agonists are therapeutically important because of their dual nature. For example, the μ-opiate receptor partial agonist buprenorphine is less effective than morphine in stimulating the μ-opiate receptor and antagonizes the actions of morphine at this receptor. It is used for treatment of opiate addiction because it blocks the actions of morphine and heroin at the μ-opiate receptor to allow for the addictive drugs to be tapered off while producing some stimulation itself, thereby preventing a full-blown withdrawal reaction.

Inverse agonists are also able to block the effects of full agonists at GPCRs, but they also induce opposite effects on the same GPCR as full agonists. Thus, whereas norepinephrine or isoproterenol will stimulate the β-adrenergic receptor to increase adenylyl cyclase activity, inverse agonists would bind to this receptor to decrease adenylyl cyclase activity. The inherent activity of an inverse agonist is dependent on the receptor having some level of constitutive basal activity (Kenakin T, Bond R, Bonner T: Definition of pharmacological receptors. Pharmacol Rev 1992; 44:351-362).

In fact, as described above, most recombinant GPCRs overexpressed in cell lines produce constitutive basal activity that is caused in part by the generation of homodimers. Under these conditions, compounds that might otherwise be considered neutral antagonists produce inverse agonism. Inverse agonists may also be useful in pathological conditions where GPCRs undergo constitutive activity in vivo either because mutations cause the constitutive activity or because the receptors become overexpressed.

Because elevated basal activity of a GPCR is needed to see inverse agonism, activity of an identified inverse agonist may manifest in vivo as an inverse agonist or a neutral antagonist. In certain aspects of the invention, the desired inverse agonism activity of a composition may need to be confirmed through use of other assays (e.g., in vivo assays that measure the desired effect on a biological process).

### Exemplary Chimera Compositions of the Invention

Known GPCR binding proteins can be used in the design of the chimera compositions, and the second peptide component of the composition can correspond to established GPCR signaling complex partners such as CRF-BP fused and the IGF-BPs (IGF-BP1 through IGF-BP6 and Cyr61, also known as IGF-BP10 or CCN1). This overall approach of the invention, however, is not limited to known binding proteins, and other N-terminal peptides or other molecules can be used to generate GPCR chimeras. This includes the use of any other molecules known to be part of a GPCR signaling process and which modulate physiological processes through a GPCR signaling complex. Thus, the methods of the invention and compositions of the invention include the addition to the chimera of such diverse molecules as secreted proteins, extracellular matrix molecules such as neurexins, tyrosine kinase receptors, cadherins and integrins to N-terminus of GPCRs.

In one specific example, the second peptide of the chimera corresponds to one or more insulin growth factor (IGF) binding proteins (IGF-BPs). A subset of GPCRs, such as the thrombin receptor (PAR1), mediate cell proliferation in human astrocytoma cells, an effect mediated via Cyr61. It has been shown that Cyr61 interacting with the integrin receptors, alpha5 and beta1 integrin, induce DNA synthesis in astrocytoma cells (Walsh et al., 2008). The efficacy of thrombin in inducing DNA synthesis is reduced in the absence of Cyr61, suggesting that activation of the GPCR, PAR1 partly requires Cyr61 to induce DNA synthesis in astrocytoma cells.

Thus, in a specific example, chimera compositions can have components that correspond to Cyr61 and PAR1 to identify novel molecules that inhibit the interaction of Cyr61 and PAR1 with the goal of targeting tumor cells to inhibit cell proliferation.

The insulin-like growth factors (IGFs) are potent mitogens. IGFs interact with the extracellular matrix protein (ECM) such as vitronectin (VN) via IGF-BP -2,-3,-4 and -5 (Kricker et al., Endocrinology; 145:193 (2003); Schneider M R et al., Endocrinology; 172: 423-440 (2002)). IGF interacting with IGF-BP and VN stimulate migration and proliferation in a variety of cells including human breast epithelial, osteoblast-like, and skin and corneal epithelial cell lines. Chimera compositions can contain peptides that correspond to IGF-BP (2-6) and/or vitronectin to identify novel molecules that stimulate the interaction of integrin receptors and specific chemokine receptors with the goal of enhancing the ability of leukocytes to target sites of inflammation.

In other aspects, interphotoreceptor retinoid-binding protein (IRBP), which is present in the extracellular space between photoreceptors and the RPE, can be fused to GPCRs for the identification of molecules involved in mechanisms of light perception and transmission of visual signaling. IRBP is known to bind visual retinoids, and studies have shown that IRBP plays an insignificant role in opsin-pigment regeneration. Jin M et al., J Neurosci. 2009 Feb. 4; 29(5):1486-95. Chimera compositions that contain IRBP sequences are useful to identify novel molecules involved in such signaling and to elucidate mechanisms of light perception and the function of rod or cone photoreceptor cells.

In a more specific example, the chimera compositions of the invention modulate signaling pathways in response to light. These compositions are based on light-modulated GPCR classes such as the opsins, including but not limited to Rhodopsin, cone opsins (Photopsins) Melanopsin, Pineal Opsin (Pinopsin), Vertebrate Ancient (VA) opsin, Parapinopsin (PP) Opsin, Extraretinal (or extra-ocular) Rhodopsin-Like Opsins (Exo-Rh), Encephalopsin or Panopsin, Teleost Multiple Tissue (TMT) Opsin, Peropsin or Retinal pigment epithelium-derived rhodopsin homolog, Retinal G protein coupled receptor, and Neuropsin or kallikrein-related peptidase 8. In addition to these naturally-occurring opsins, the designer OptoXRs, which comprise the N-terminal domain of an opsin and the intracellular domain of another GPCR (Airan R D, Nature. Apr 23; 458(7241):1025.-2009 Mar. 18. [Epub ahead of print]), can be used as the basis for compositions of the invention. The chimera compositions feature such opsin sequences fused to peptide domains involved with light-based modulation, including but not limited to the light activating domains of the channelrhodopsins, a subfamily of opsin proteins that function as light-gated ion channels. Three channelrhodopsins are currently known: Channelrhodopsin-1 (ChR1), Channelrhodopsin-2 (ChR2), and Volvox Channelrhodopsin (VChR1).

In addition to the traditional view of GPCR signaling, it is now clear that GPCRs also participate in non-GPCR-protein interactions. The discovery that GPCRs exist in complexes with other proteins, such as tyrosine kinase and integrin receptors, within specialized domains in cells suggest that GPCR signaling may be modulated by each of these different protein interactions. The advantage of a signaling complex localized to a cell microdomain is that it provides a mechanism to have both spatial and temporal resolution of signaling, in reaction to different stimuli in the extracellular environment. GPCR ligand binding sites are changed as a consequence of interactions with binding proteins, suggesting that these different types of proteins interacting with GPCRs will modulate the affinity of GPCR ligands for GPCRs by altering G protein coupling affinity states.

Certain GPCR ligands are potent cellular growth factors, induce cell proliferation by acting synergistically with tyrosine kinase receptors and play a role in cell growth and differentiation in a host of different disorders including cardiac hypertrophy and tumorigenesis. The second peptide component of the compositions of the invention may correspond to such growth factors or, alternatively, to these kinase receptors that interact with such growth factors in GPCR-mediated signaling events. Examples of certain growth factors and kinase receptors are provided below.

A large number of studies demonstrate that GPCR ligands transactivate EGF tyrosine kinase receptors via ectodomain shedding of the EGF receptor. A key pathway involved in mitogenic GPCR signaling is the extracellular signal-regulated kinase (Erk1/2) mitogen activated protein (MAP) kinase. The duration and intensity of ERK pathway activation is important in defining biological outcomes such as proliferation, differentiation and transformation.

MAP kinase activation has been implicated in a diverse array of biological effects, however, a convergence of signals resulting from GPCRs and growth factors on this pathway often leads to physiological responses associated with activation of the mitogenic pathway. EGF-R transactivation by GPCRs is important for prolonging the Erk1/2 signal in response to GPCR ligands. GPCRs frequently couple to two or more G proteins; it has been proposed that receptors exist in three different conformational states; (1) an inactive state (absence of agonist), (2) a state that can activate G12; and (3) a state that activates Gq (Kobilka, 2007). Protein-protein interactions between GPCRs and tyrosine kinase receptors will induce a conformational state of the GPCR to favor activation of the MAP-kinase pathway. Erk1/2 activation by GPCRs involves crosstalk with classical tyrosine kinase receptors or focal adhesion kinases that scaffold the assembly of a Ras activation complex. Highly organized signaling complexes determine the location, duration and ultimate function of GPCR-stimulated MAP kinase activity.

In specific examples, EGF-R (ErbB2) complexes with both Class A and B GPCRs in cardiac myocytes and deletion of ErbB2 prevents GPCR ligand signaling to Erk1/2 (Negro A et al., Proc Natl Acad Sci USA. 2006 Oct. 24; 103(43):15889-93. Epub 2006 Oct. 16. 2006). One interpretation of this data is that ErbB2 alters the conformational state of the GPCR and prevents GPCR ligand to stimulate Erk1/2 activity. Chimeras of ErbB2-GPCR (class A-C) in cells can provide a mechanism to identify novel molecules that stimulate or inhibit Erb-B1-GPCR activation of Erk1/2. Activation of mGluR5, a Class C GPCR, induces association of the EGF receptor, ErbB1 and induces phosphorylation of MAP kinase in cultured rat astrocytes (Peavy et al., J Neurosci. 2001 Dec. 15; 21(24):9619-28. 2001).

Leukocytes circulating in the blood are selectively recruited to specific target sites through a process of adhesive interactions and activation signals. In vitro studies have shown that rapid triggering of integrin adhesiveness is transduced by GPCRs occupied by immobilized, endothelial-presented chemokines (Laudanna and Alon, Thromb Haemost. 2006 January; 95(1):5-11. 2006). The chemokines capable of triggering integrin-mediated leukocytes arrest appear to function when located near an integrin ligand. The ability of chemokines to rapidly trigger integrin adhesiveness depends both on the type of GPCR they bind to and the magnitude of the signal generated. This suggests that binding protein may exist for chemokines, such as the IGF's, that provide a mechanism of presenting chemokines to their cognate chemokine receptors (GPCR) to provide leukocytes with specific ways to control adhesiveness. These different type of proteins modulate the affinity of GPCR ligands alter GPCR-G protein coupling affinity states, and such interactions can be reproduced in part using the compositions of the invention.

Chimera compositions can thus be made of integrin and chemokine receptors to identify novel molecules that stimulate the interaction of integrin receptors and specific chemokine receptors with the goal of enhancing the ability of leukocytes to target sites of inflammation. Second peptides corresponding to integrins can be fused to the N-terminus of first peptides corresponding to chemokine receptors to determine whether binding proteins can modulate GPCR signaling in such systems.

In another example, the disease to be treated is an autoimmune disease, e.g., rheumatoid arthritis, Crohn's disease and multiple sclerosis. Such disorders display chronic inflammatory reactions, with neutrophils in the inflamed tissue expressing the chemokine receptors CXCR3 and CCR5 and the ligands CXCL9, 10 and 11, and T cells in the inflamed tissue expressing CCL5. CCL11 is a known in vitro agonist of CXCR3 function, and CXCL9, 10 and 11 are known to inhibit CCR3 a potential target for intervention in allergic disease. By using compositions with sequences that correspond to these receptors and ligands, new drug candidates for autoimmune disease may be identified.

In specific aspects of the invention, the chimera peptides are indirectly fused, i.e. linked by other, intervening amino acids and/or peptide structures. The sequences or ligands are polymers having monomers based on known protein interaction domain structures which have specific physical structures. Such intervening sequences include, but are not limited to, non-functional linker sequences, e.g., sequences that aid in construction of the chimera or that serve as epitope tags or other identifiers; smaller functional proteins (e.g., hormones and ligands) that interact with the other components of the chimera compositions; domain-based peptides, e.g., sequences based on known protein domains that can be used to provide appropriate spacing between the peptides of the composition, to stabilize the composition, to provide appropriate localization of the compositions, and the like; and larger proteins with desirable traits, such as fibronectin or vitronectin. The intervening peptide sequences can thus vary from an amino acid linker to an epitope tag sequence to a fluorescent identifier to a short functional peptide to a known protein domain, or any combination thereof.

In specific aspects, intervening sequence can comprise a single or multiple monomer domains, including monomers with variations of the same domain structures, or combinations of monomer domains that have similar specificity, or variations of different classes of monomer domains selected based on the structure and desired spatial relationships of the other components of the chimera compositions. Examples of conserved domains and repeats that can be used as intervening sequences in the present invention include, but are not limited to, those found in the EMBL SMART database, which can be accessed at http://smart.embl-heidelberg.de/smart/domain_table.cgi.

### Identification of Ligands for Orphan Receptors

The chimera compositions of the invention provide a unique opportunity to identify the native ligand(s) for orphan GPCRs. Numerous GPCRs without a known endogenous ligand have been identified, and many of these molecules are interesting targets for pharmacological intervention. By using compositions of the invention based on such orphan receptors and one or more potential binding proteins predicted to associate with these receptors, ligands and other modulating proteins may be identified for these orphans. The chimera compositions can be uniquely designed for identification of ligands for individual orphan receptors or groups of related orphan receptors. Moreover, the assays of the invention are particularly well suited for screening of large numbers of potential ligands, e.g., by testing a large peptide library comprising potential ligands against the chimera compositions of the invention.

Exemplary Therapeutic Uses for Compositions of the Invention and/or their Binding Partners.

In a particular aspect of the invention, the compositions of the invention are used to identify binding partners that are drug candidates for treatment of neurological conditions associated with particular biological processes. The following are exemplary chimera compositions and neurological conditions that may be amenable to therapeutic intervention using binding partners of the compositions of the invention. The invention is not meant to be limited to such examples; rather, use of a single example is presented so as to better elucidate the aspects of the invention without obscuring the basic elements of the novelty of the invention. This specific example demonstrates the mechanistic approach of the invention and is not meant in any way to limit the invention's scope.

It was previously discovered that an inhibitor of the CRF-binding protein (CRF-BP) blocks the effects of CRF, which indicates that CRF-BP is necessary for CRF to potentiate NMDAR currents. See US Pat. App. No. 20060024661, which is incorporated by reference in its entirety. Thus, in a specific aspect, the invention provides a chimera composition having CRF-BP fused to the N-terminus of CRFR2, a Class B GPCR. This composition can be used as a research tool to identify not only molecules which selectively inhibit signaling through CRFR2, but also molecules which selectively potentiate or activate signaling through CRFR2.

The use of such a chimera composition is also powerful in that it can differentiate interactions between highly related GPCRs, in this case CRFR1 and CRFR2, and their selective binding partners. This will open new avenues for discovery and development of potential therapeutic targets in disorders where CRF and/or dopamine levels are decreased. For example, identification of binding partners which modulate CRFR2 and not CRFR1 may lack the anxiogenic properties of other CRF-like peptides that bind selectively to CRFR1. Examples of neurological conditions that may benefit from the use of the compositions of the invention include the following:

### Substance Abuse.

A wide variety of studies have shown that stressors increase vulnerability for drug self-administration in rodents not previously exposed to psychostimulants (Piazza et al., Brain Res. 1991 Dec. 13; 567(1):169-74) as well as demonstrate that CRF plays an important role in mediating behaviors produced by many drugs of abuse (see, e.g., Sarnyai et al., Pharmacol Rev. 2001 June; 53(2):209-43., Shalev et al., Pharmacol Rev. 2002 March; 54(1):1-42; Sinha, Ann N Y Acad Sci. 2008 October; 1141:105-30.; Bonci and Borgland, Neuropharmacology. 2009;56 Suppl 1:107-11. Epub 2008 Jul. 24; Koob, Neuropharmacology. 2009;56 Suppl 1:18-31. Epub 2008 Aug. 7). The role of the CRF-BP in mediating drug-dependent behaviors is unknown, but disruption of the CRF-CRF-BP interaction reduces stress-induced relapse to drug seeking (Wang et al., J Neurosci. 2007 Dec. 19; 27(51):14035-40).

### Depression.

A variety of human and rodent studies have shown significant changes in CRF levels in patients suffering from depression (reviewed in Todorovic et al., Neurosci Biobehav Rev. 2005; 29(8):1323-33. Epub 2005 Aug. 15; Van Den Eede et al., Ageing Res Rev. 2005 May; 4(2):213-39; Dunn and Swiergel, Ann N Y Acad Sci. 2008 December; 1148:118-26; Goel and Bale, J Neuroendocrinol, 21-415-420, 2009 Jan. 30. [Epub ahead of print]. For example, hyperactivity of the CRF system has been associated with major depressive disorder in patients (reviewed in the studies above). Furthermore, increased CRF-like immunoreactivity has been reported in CSF of patients with depression.

### Alzheimer's Disease.

Many studies have suggested a role for a CRF system imbalance in Alzheimer's disease (e.g. Behan et al., Nature. 1995 Nov. 16; 378(6554):284-7; Behan et al., J Neurochem 1997 May; 68(5):2053-60.1997; Hogan et al., Neuropsychiatr Dis Treat. 2007 October; 3(5):569-78., and references therein). For example, a study in which cerebrospinal fluid (CSF) CRH-immunoreactivity was measured correlated a lower CSF CRH-immunoreactivity with a greater cognitive impairment in Alzheimer patients (reviewed in Gallagher et al., Eur J Pharmacol. 2008 Apr. 7; 583(2-3):215-25. Epub 2008 Feb. 1). Behan et al., (J Neurochem. 1997 May; 68(5):2053-60) demonstrated that in patients suffering from Alzheimer's disease there are dramatic reductions in human CRH concentrations and reciprocal increases in CRH receptor density in the cortex.

### Obesity.

The CRF signaling pathways play a key role in the alteration of feeding behavior (Zorrilla et al., Trends Pharmacol Sci. 2003 August; 24(8):421-7; Bale and Vale, Annu Rev Pharmacol Toxicol. 2004;44:525-57; Dallman et al. J Physiol. 2007 Sep. 1; 583(Pt 2):431-6 Epub 2007 Jun. 7; Dallman et al. Prog Brain Res. 2006;153:75-105). Glucocorticoids chronically increase palatable food intake, which in turn increases abdominal fat deposits and circulating insulin levels, both of which negatively correlate with CRF mRNA expression in the PVN (Warne, Mol Cell Endocrinol. 2009 Mar. 5; 300(1-2):137-46. Epub 2008 Oct. 15). Several reports have established that CRF, Ucn 1, Ucn 2, and Ucn 3 injected into the brain reduce food intake in rodents through CRF1 and CRF2 receptor-dependent mechanisms that mediate, respectively, the acute (first hour) and delayed (3-6 h) anorexic responses (Cullen et al., Endocrinology. 2001 March; 142(3):992-9; Richard et al., Eur J Pharmacol. 2002 Apr. 12; 440(2-3):189-97.; Inoue et al., J Pharmacol Exp Ther. 2003 April; 305(1):385-93.; Fekete et al., Front Neuroendocrinol. 2007 April; 28(1):1-27. Epub 2006 Nov. 2).

### Schizophrenia and Other Psychiatric Conditions.

A wide variety of studies have highlighted the importance of developing molecules targeted at the CRF system as potentially promising therapeutics against a variety of psychiatric conditions (Herringa et al., Neuropsychopharmacology. 2006 August; 31(8):1822-31. Epub 2006 February 82006). For example, studies have reported increased CSF levels of CRF in subjects with depression or obsessive-compulsive disorders (Arborelius et al, J Endocrinol. 1999 January; 160(1):1-12.; Mitchell, Neurosci Biobehav Rev. 1998 September; 22(5):635-51). In depressed individuals, CSF CRF levels also tend to normalize after successful SSRI treatment, suggesting that high CSF CRF should be considered a state-dependent finding, rather than a trait marker for depression (Mitchell, 1998, supra). Additionally, an imbalance of the CRF-CRFBP system has been reported both in patients suffering from either schizophrenia or bipolar disorders (Herringa et al., 2007, supra; Goel and Bale, 2007, supra.

### Infectious Agents.

A number of infectious agents, including many viruses (e.g., HIV) and protein-based agents (e.g., prions or the β-amyloid peptide) require cell surface signaling complexes for the infection of mammalian cells. These infectious agents can use these cell surface proteins to infect, internalize and effectively highjack the normal functions of these cells in a receptor-specific manner Compositions of the invention may thus comprise an infectious peptide fused to a GPCR receptor to identify agents that may disrupt this interaction and thus prevent or halt the activity of the infectious agent.

For example, Human Immunodeficiency Virus 1 (HIV-1) requires a chemokine receptor in addition to CD4 for efficient entry into cells. Simmons G et al. Immunol Rev. 2000 October; 177:112-26. Circulating isolates of HIV have shown extremely expanded GPCR usage beyond the initially identified receptors CCR5 and CXCR4, and CPCRs that can function as coreceptors include CCR1, CCR3, CCR5, CCR8, CXCR4, D6, FPRL1, and GPR1 as coreceptors. Shimizu N et al., AIDS. 2009 Mar. 19. In another example, peptide domains derived from the envelope proteins of human immunodeficiency virus type 1 (HIV-1), the human acute phase protein serum amyloid A, the 42 amino acid form of beta amyloid peptide and a 21 amino acid fragment of human prion selectively activate the high-affinity fMLF receptor FPR and/or its low-affinity variant FPRL1. Le Y, Int Immunopharmacol. 2002 January; 2(1):1-13; Le Y et al., J Immunol. 2001 Feb. 1; 166(3):1448-51. The interaction of these GPCRs with these peptides provides a major pharmaceutical target for disruption of the binding of these agents to GPCRs, and may identify drug candidates for the prevention and/or attenuation of the effects of such viral and protein-based agents. Thus, chimera compositions of the invention comprising both a GPCR and a fused infectious agent may be invaluable research tools for discovery of drugs that specifically interaction of infectious agents with such receptor complexes

### Examples

The following examples are put forth so as to provide those of ordinary skill in the art with a complete disclosure and description of how to make and use the present invention, and are not intended to limit the scope of what the inventors regard as their invention, nor are they intended to represent or imply that the experiments below are all of or the only experiments performed. It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

Efforts have been made to ensure accuracy with respect to numbers used (e.g., amounts, temperature, etc.) but some experimental errors and deviations should be accounted for. Unless indicated otherwise, parts are parts by weight, molecular weight is weight average molecular weight, temperature is in degrees centigrade, and pressure is at or near atmospheric.

### Example 1

### Cloning and Production of CRF-BP_CRFR2 Chimeras

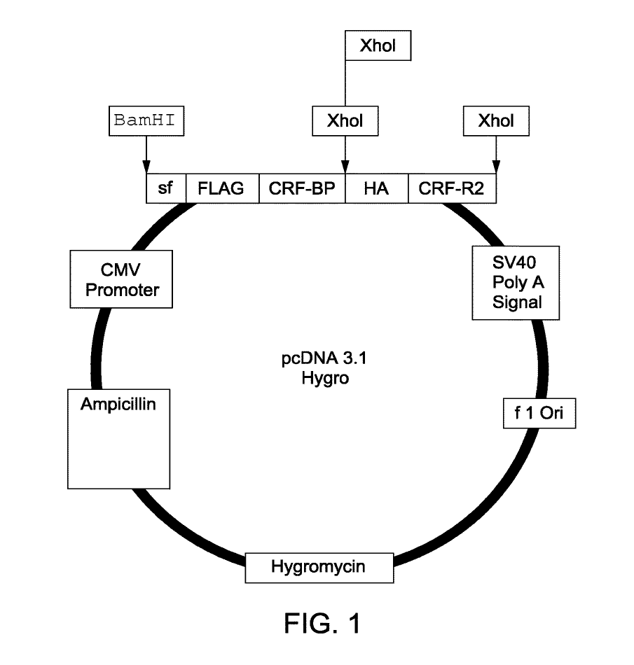

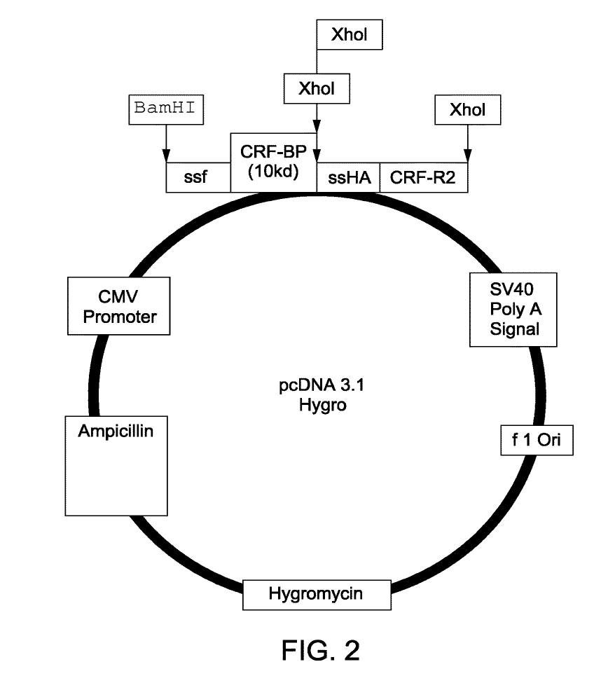

Human CRF-BP_CRFR2 chimeras were produced by initial cloning of the first GPCR peptide and second signaling complex peptide into a pcDNA3.1 vector. The map of the vector produced for the expression of the full-length CRF-BP CRFR2 chimera is shown in FIG. 1. The map of the vector produced for the expression of the chimera comprising a 10 Kd fragment of CRF-BP with CRFR2 is shown in FIG. 2. The CRF-BP (10 Kd) fragment is comprised of 88 amino acid residues (A₂₃₅ to L₃₂₂): AGCEGIGDFVELLGGTGLDPSKMTPLADLCYPFHGPAQMKVGCDNTVVRMVSS GKHVNRVTFEYRQLEPYELENPNGNSIGEFCLSGL (SEQ ID NO:1). Construction of these plasmids is as described below.

A pcDNA13 vector (Invitrogen, Carlsbad, Calif.) was digested with BamHI, and XhoI (NEB, Ipswich, Mass.), and the digested DNA was run on a 1.2% agarose gel at 70 volts for 50 min and the desired fragment purified using a Qiagen (Valencia, Calif.) Gel Extraction Kit.

CRFBP (FL) fragment was excised using BamHI and XhoI from Origene and was gel purified using a Qiagen gel extraction kit. The fragment was eluted in water. A pcDNA3.1_Hygro vector with FLAG tag was digested with BamHI and XhoI. This vector fragment was then treated with CIP (Calf intestinal phosphate) to prevent self ligation. This fragment was also gel purified using Qiagen gel extraction kit and eluted in water. Following elution, the fragment was combined with the BamHI and XhoI digested vector, and these components were ligated using T4 DNA ligase (NEB).

Chemically competent E. Coli cells (Invitrogen, Carlsbad, Calif.) were transformed with the pcDNA3.1_Hygro/ CRFBP construct, plated on LB plates, and allowed to incubate overnight at 37° C. The cells were then cultured in 5 ml LB, and the vector was isolated via a standard plasmid preparation using a Qiagen (Ventura, Calif.) mini prep kit. The sequence of the plasmids was confirmed by restriction endonuclease digestion and sequencing.

The CRFBP (10 kd) fragment was amplified using FLAG-CRF-BP (FL)_Hygro plasmid DNA as the template and CRFBP10_BamHI_For and PCR_XhoI_Rev primers ATAGGATCCGGCAGGTTGCGAGGGAATAG (forward) (SEQ ID NO:2) and ATACTCGAGTAGAAGGCACAGTCGAGG (reverse) (SEQ ID NO:3). The XhoI and XbaI digestion sites in the primers are shown in bold. 1.5 μl (approximately 150 ng) of template DNA was used in the PCR reaction, and 2.5 μl of each primer was used at a concentration of 5 μM. The DNA was amplified using 0.2 μl Highfidelity Taq at 5 u/μl in a total volume of 50 μl. The PCR conditions used were 30 cycles of 94° C. for 30 seconds, 59° C. for 30 seconds and 68° C. for 30 seconds, followed by a final extension at 68° C. for 5 minutes. The PCR products were then purified using PCR purification kit from Qiagen (Valencia, Calif.).

The PCR fragment was purified using a Qiagen PCR cleanup kit and eluted in water. Later the PCR fragment was digested with BamHI and XhoI and was again purified using a Qiagen PCR cleanup kit and eluted in water. A pcDNA3.1_Hygro vector with FLAG tag was digested with BamHI and XhoI and was treated with CIP (Calf intestinal phosphate) to prevent self ligation. This fragment was gel purified using Qiagen gel extraction kit and eluted in water. Following elution, the two DNA components were ligated using T4 DNA ligase (NEB).

Chemically competent E. Coli cells (Invitrogen, Carlsbad, Calif.) were transformed with the ligated FLAG-CFR-BP vectors, plated on LB plates, and allowed to incubate overnight at 37° C. The cells were then cultured in 5 ml LB, and the vectors isolated via a standard plasmid preparation using a Qiagen (Ventura, Calif.) mini prep kit. The sequence of the plasmids and the desired orientation for expression was confirmed by restriction endonuclease digestion and sequencing.

DNA encoding the CRFR2 protein was amplified using the ssHA CRFR2_Zeo plasmid DNA template and the following primers: ATACTCGAGTATCCTTACGACGTGCCTGA(forward) (SEQ ID NO:4) and ATATCTAGAAATTCGCCCTTGTCGACTC (reverse) (SEQ ID NO:5). The XhoI and XbaI digestion sites in the primers are shown in bold. 1.5 μl (approximately 150 ng) of the template DNA and 2.5 μl of each primer at a concentration of 5 μM were used in the PCR reaction. The DNA was amplified using 0.2 μl Highfidelity Taq at 5 u/μl in a total volume of 50 μl. The PCR conditions used were 30 cycles of 94° C. for 30 seconds, 59° C. for 30 seconds and 68° C. for 30 seconds, followed by a final extension at 68° C. for 5 minutes. The PCR products were then purified using PCR purification kit from Qiagen (Valencia, Calif.).

The purified HA CRFR2 DNA and the FLAG-CFR-BP (FL)_Hygro vector or the FLAG-CFR-BP (10 kd)_Hygro vector were both digested with XhoI and XbaI (NEB, Ipswich, Mass.), and the desired fragments were gel purified using a Qiagen (Valencia, Calif.) Gel Extraction Kit. The digested PCR product and vector DNA were ligated using T4 DNA ligase. The ligation reaction included 1 μl purified vector DNA, 2 μl purified CRFR2 DNA, 2 μl 10× ligase buffer, 1 μl T4 DNA ligase (NEB, Ipswich, Mass.) and 14 μl water. The ligation reaction mixture was incubated at 16° C. overnight, followed by incubation at 65° C. for 20 minutes. It was stored at 4° C. until further use as described below.

Chemically competent E. Coli cells (Invitrogen, Carlsbad, Calif.) were transformed with the ligated FLAG-CFR-BP (FL)_HA CRFR2_Hygro vector or the FLAG-CFR-BP (10 kd)_HA CRFR2_Hygro vector, plated on LB plates, and allowed to incubate overnight at 37° C. The cells were then cultured in 5 ml LB, and the vector isolated via a standard plasmid preparation. The sequence of the plasmids and the desired orientation of the insert for proper expression was confirmed by restriction endonuclease digestion and sequencing.

### Example 2

### Transfection of Human Cells with the Expression Plasmid

The plasmids containing the CRF-BP_CRFR2 fusion chimeras were then transfected into HEK 293 cells for expression of the protein, confirmation of the appropriate insertion into the membrane, and functionality of the chimera in mammalian cells.

One day prior to transfection, HEK 293 cells were placed in a 12 well plate with DMEM with 10% fetal bovine serum (FBS) in each of the wells. Immediately prior to transfection, each well was examined to confirm approximately 90-95% confluency of the cells in the wells. The DMEM was carefully aspirated from the wells, and replaced with fresh DMEM/10% FBS.

The final DNA plasmid preparations created in Example 1 were then diluted in 100 μl of Opti-MEM (reduced serum) and gently mixed. Lipofectamine 2000 which had been likewise diluted in 100 μl of Opti-MEM was incubated at room temperature for 5 minutes, and then combined with the diluted DNA. This was mixed gently and incubated at room temperature for 20 minutes.

Approximately 200 μl of the Lipofectamine 2000-DNA complexes were added to each well to a total volume of 1.2 ml/well. The well contents were mixed gently by rocking the plate back and forth, and incubated at 37° C. for 5 hours. After incubation media was replaced with fresh DMEM/10% FBS. The cells were then selected by growing the cells in 10% FBS in DMEM cell media with hygromycin (200 μg/ml) selection reagent. From these initial experiments, six clones were isolated that demonstrated consistent and easily measurable expression of the chimera proteins in the transfected HEK 293 cell lines as determined by Western Blot analysis. The expression of the chimera clones in the cell lines was determined using CRF-BP mouse monoclonal antibody against full length CRF-BP (human origin). 50 μg of cell lysate was loaded per well on 4-20% Tris-Glycine gel from Invitrogen (Carlsbad, Calif.).

These chimeras were further tested for functionality by measuring their activity in activation and inhibition assays performed as described in the following examples.

### Example 3

### Signaling Activation Through the CRF-BP_CRFR2 Chimeras

The mammalian cells expressing the isolated chimera constructs were tested for the ability of the chimera proteins to activate intracellular calcium release via signaling through Gq. HEK 293 cells of Example 2 expressing the constructs of Example 1 were grown in 10% FBS in DMEM cell media with hygromycin (0.4%) selection reagent. Cells were plated out in 96-well plates (40 000 cells/well) in FBS/DMEM media. On the following day, cells were serum starved (1% FBS in DMEM; 100 μl/well) for 2 hours prior to testing.

Cells were loaded with diluted FLIPR™ dye (100 μl) for one hour prior to testing with CRF. The selected hygromycin resistant cells were plated in 96-well clear bottom black microplates at a density of approximately 40,000 cells/well, in DMEM/10% FBS media. One vial of Ca²⁺ dye (Molecular Devices) was diluted in 10 ml of 1× Washbuffer [10 mL 10× Hank's Balanced Salt Solution, 2 mL HEPES 1 M, 87 mL distilled water, 1 mL Probenecid 250 mM (71 mg dissolved in 1 ml 1N NaOH), Set pH to 7.4], and on the day of the assay, 100 μl of the diluted Ca²⁺ dye was added to each well containing the cells, for a total volume of 200 μl/well. The plates were incubated for 60 minutes at 37° C.

The cells expressing the full-length CRF-BP_CRFR2 chimera were then treated with either CRF or with the CRF fragment (6-33), LVSAGVLLVALLPCP PCRALLSRGPVPG (SEQ ID NO:6) in a range of concentrations (1 pM-10 μM) added to wells in a volume of 50 μL per well. For the inhibition experiments, CRF (6-33) (10 pM-100 μM) was added to the wells in a volume of 2 μl per well) and incubated for 30 min prior to measurement of the activation of a constant concentration of CRF (1 μM).

A FlexStation (Molecular Devices) fluorometric imaging plate reader was used to measure changes in intracellular Ca²⁺. The plates were placed in the FlexStation for the assay. The machine was used in Flex mode, and the fluorescent intensity was measured from the bottom with the excitation at 485 nm and emission at 525 nm for 120 seconds at 21° C.

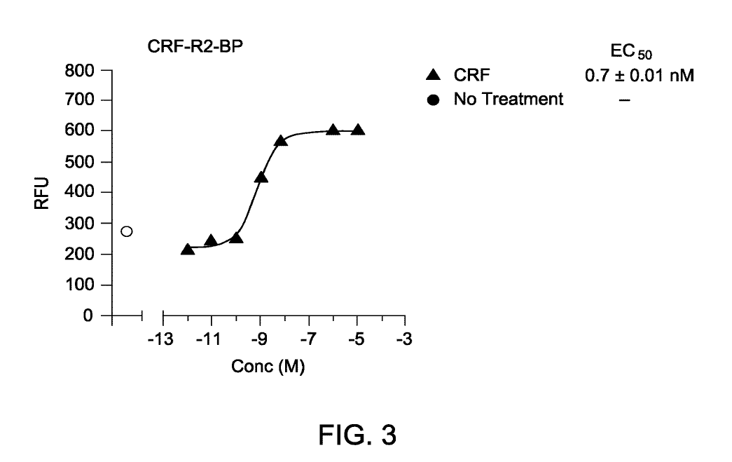

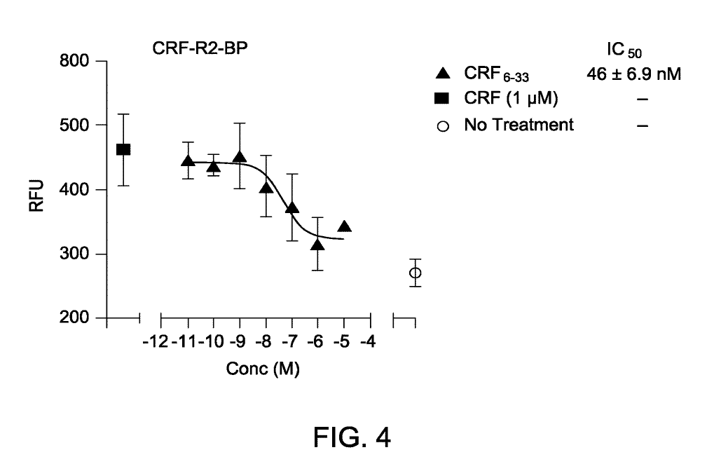

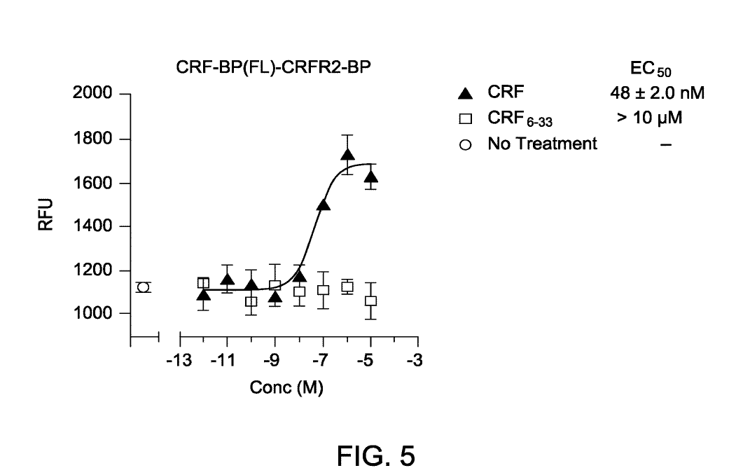

The results of the FLIPR experiments detecting the levels of intracellular calcium induced by activation of the chimera with CRF are shown in FIGS. 3-5. As shown in FIG. 3, the heterodimer chimera composition displayed levels of CRF-induced (1 pM-10 μM) intracellular calcium release in the HEK293 cells that increased as the concentration of CRF was increased. The CRF fragment, CRF₆₋₃₃ (1 pM-10 μM), did not stimulate intracellular calcium release in chimera expressing cells (FIG. 4). FIG. 5 shows the juxtaposition of the results obtained using the full-length CRF molecule versus use of the CRF₆₋₃₃ fragment. Results are expressed as the mean (±SEM) relative fluorescent units (RFU), calculated as agonist-induced maximum Ca²⁺ peak/cell number×1000.

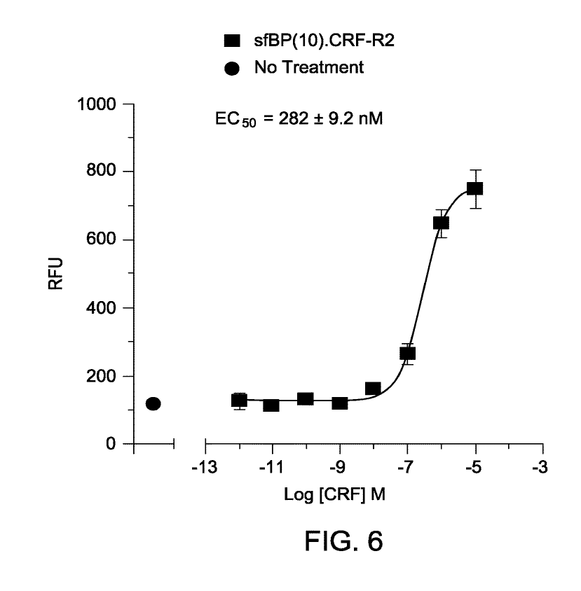

The cells expressing the CRF-BP(10 Kd)_CRFR2 chimera were then treated with CRF, in a range of concentrations (1 pM-10 μM) added to wells in a volume of 50 μL per well and incubated for 30 min prior to measurement of the activation of a constant concentration of CRF (1 μM). The chimera compositions with the CRF-BP(10 Kd) displayed even more robust signaling than the full-length CRF-BP_CRFR2 chimera, as shown in FIG. 6. Results are expressed as the mean (±SEM) relative fluorescent units (RFUs), calculated as agonist-induced maximum Ca²⁺ peak/cell number×1000.

The experiments demonstrate that CRF interacts with both the CRF-BP_CRFR2 and CRF-BP(10 Kd)_CRFR2 chimeras, and is able to signal though these chimera constructs to control intracellular calcium release, indeed more effectively than CRF at CRFR2 alone. In addition, the level of activation was shown to correlate with the expression level of the chimera in the cell line (data not shown). These results suggest that CRF-BP is at the surface and modulating CRFR2. In addition, the treatment of the cells with CRF₆₋₃₃ demonstrated an inhibition of signaling compared to treatment with CRF, as demonstrated in FIGS. 4 and 5.

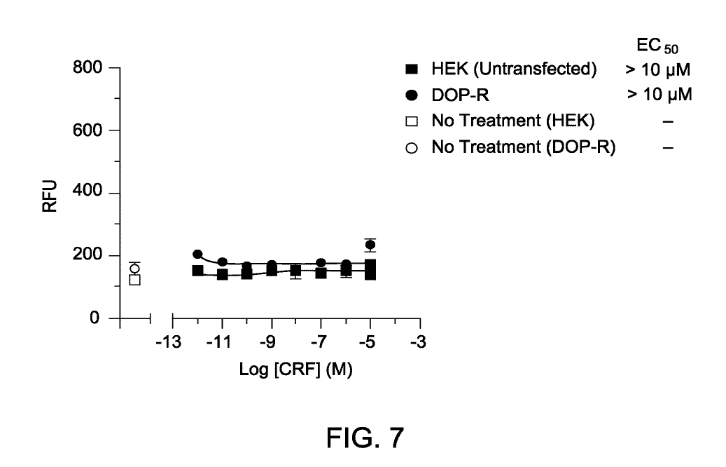

As a negative control, the effect of CRF on HEK 293 cells that had not been transfected or which expressed a recombinant dopamine receptor was measured. Cells were treated with CRF as described above, and did not display any significant signaling (FIG. 7).

Though this particular assay was performed on a smaller scale in 96 well plates, the calcium assay performed is scalable to a 384 well format to facilitate screening of test agents that modulate CRF signaling through CRFR2 in vivo.

### Example 4

### Cloning and Production of a CRF-BP NK₁R Chimera

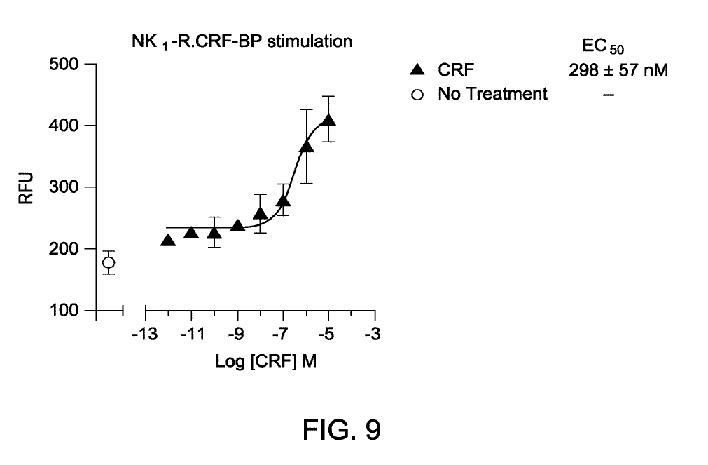

The neurokinin-1 receptor (NK₁R) is a class A GPCR of the tachykinin receptor sub-family. A human CRF-BP_NK₁R chimera was produced to demonstrate that the GPCR chimera compositions of the invention reflect a more general approach applicable to all GPCR classes. The peptide and second signaling complex peptide were cloned into a pcDNA 3.1 vector for production of the chimera composition, as illustrated in FIG. 9. Construction of this plasmid is as described below.

A pcDNA13 vector (Invitrogen, Carlsbad, Calif.) was digested with BamHI and XhoI (NEB, Ipswich, Mass.), and the digested DNA was run on a 1.2% agarose gel at 70 volts for 50 min and the desired fragments were purified using a Qiagen (Valencia, Calif.) Gel Extraction Kit. Following elution in water, the two DNA components were ligated to insert the His-tag oligonucleotide into the recircularized vector.

Chemically competent E. Coli cells (Invitrogen, Carlsbad, Calif.) were transformed with the vector, plated on LB plates, and allowed to incubate overnight at 37° C. The cells were then cultured in 5 ml LB, and the vector isolated via a standard plasmid preparation using a Qiagen (Ventura, Calif.) mini prep kit. The sequence of the plasmids was confirmed by restriction endonuclease digestion and sequencing.

DNA encoding the NK₁R protein was amplified using human ssHA NK1_Zeo and the following primers to the pcDNA 3.1 vector: ATATCTAGATATCCTTACGACGTGCCTGA(forward) (SEQ ID NO:7) and ATATCTAGAAATTCGCCCTTGTCGACTC (reverse) (SEQ ID NO:5). An XbaI digestion site in the primers is shown in bold. 1.5 μl (approximately 150 ng) of human cDNA was used as template DNA in the PCR reaction, and 2.5 μl of each primer was used at a concentration of 5 μM. The DNA was amplified using 0.2 μl High fidelity Taq at 5 u/μl in a total volume of 50 μl . PCR conditions were 30 cycles of 94° C. for 30 seconds, 59° C. for 30 seconds and 68° C. for 30 seconds, followed by a final extension at 68° C. for 5 minutes. The PCR products were then purified using PCR purification kit from Qiagen (Valencia, Calif.).

The purified NK₁R DNA and the pcDNA 3.1 vector with HIS tag were both digested with XbaI (NEB, Ipswich, Mass.), and the desired fragments gel purified using a Qiagen (Valencia, Calif.) Gel Extraction Kit. The digested PCR product and vector DNA were ligated using T4 DNA ligase. The ligation reaction included 1 μl purified vector DNA, 2 μl purified NK₁R DNA, 2 μl 10× ligase buffer, 1 μl T4 DNA ligase (NEB, Ipswich, Mass.) and 14 μl water. The ligation reaction mixture was incubated at 16° C. overnight, followed by incubation at 65° C. for 20 minutes. It was stored at 4° C. until further use as described below.

Chemically competent E. Coli cells (Invitrogen, Carlsbad, Calif.) were transformed with the ligated NK₁R vector, plated on LB plates, and allowed to incubate overnight at 37° C. The cells were then cultured in 5 ml LB, and the vector isolated via a standard plasmid preparation. The sequence of the plasmids and the desired orientation of the insert for proper expression was confirmed by restriction endonuclease digestion and sequencing.

Human FLAG tagged CRF-BP DNA was then amplified using human ssf CRFBP(FL)_Hygro plasmid and the following primers: ATAAAGCTTACCATGAAGACGATCA (Forward) (SEQ ID NO:8) and ATAAAGCTTAGACAAACAGAATTCCCCGATA (Reverse) (SEQ ID NO:9). The HindIII binding site is shown in bold. 1.5 μl (approximately 150 ng) of human cDNA) was used as template DNA in the PCR reaction, and 2.5 μl of each primer was used at a concentration of 5 μM. The DNA was amplified using 0.2 μl Highfidelity Taq at 5 u/μl in a total volume of 50 μl. The PCR conditions used were 30 cycles of 94° C. for 30 seconds, 59° C. for 30 seconds and 68° C. for 30 seconds, followed by a final extension at 68° C. for 5 minutes. The PCR products were then purified using PCR purification kit from Qiagen (Valencia, Calif.).

The purified FLAG-CFR-BP PCR product and the HA tagged NK₁R_pcDNA plasmid with HIS tag were both digested with HindIII (NEB, Ipswich, Mass.), and the desired fragments were purified using a Qiagen (Valencia, Calif.) Gel Extraction Kit. The digested PCR product and vector DNA were ligated using T4 DNA ligase. The ligation reaction included 1 μl purified vector DNA, 2 μl purified CRFR2 DNA, 2 μl 10× ligase buffer, 1 μl T4 DNA ligase (NEB, Ipswich, Mass.) and 14 μl water. The ligation reaction mixture was incubated at 16° C. overnight, followed by incubation at 65° C. for 20 minutes.

The resulting plasmid contained DNA encoding FLAG tagged CFR-BP 5′ to the HA fragment with HIS tag in the middle. The NK₁R coding region is in-frame with the CFR-BP, so that any protein created using this plasmid will result in a protein having the FLAG-CFR-BP component at its N-terminus, the HIS tag, the HA components, and the NK₁R portions in frame and at the carboxy-terminus

Chemically competent E. Coli cells (Invitrogen, Carlsbad, Calif.) were transformed with the ligated CRFR2 vector, plated on LB plates, and allowed to incubate overnight at 37° C. The cells were then cultured in 5 ml LB, and the vector isolated via a standard plasmid preparation using a Qiagen (Ventura, Calif.) mini prep kit. The sequence of the plasmids and the desire orientation for expression was confirmed by restriction endonuclease digestion and sequencing.

### Example 5

### Transfection of Human Cells with the Expression Plasmid

The plasmid containing the fusion chimera was then transfected into HEK 293 cells for expression of the protein, confirmation of the appropriate insertion into the membrane, and functionality of the chimera in mammalian cells.

One day prior to transfection, HEK 293 cells were placed in a 12 well plate with DMEM with 10% fetal bovine serum (FBS) in each of the wells. Immediately prior to transfection, each well was examined to confirm approximately 90-95% confluency of the cells in the wells. The DMEM was carefully aspirated from the wells, and replaced with fresh DMEM/10% FBS.

The final DNA plasmid preparation created in Example 4 was then diluted in 100 μl of Opti-MEM (reduced serum) and gently mixed. Lipofectamine 2000 which had been likewise diluted in 100 μl of Opti-MEM was incubated at room temperature for 5 minutes, and then combined with the diluted DNA. This was mixed gently and incubated at room temperature for 20 minutes.

Approximately 200 μl of the Lipofectamine 2000-DNA complexes were added to each well to a total volume of 1.2 ml/well. The well contents were mixed gently by rocking the plate back and forth, and incubated at 37° C. for 5 hours. After incubation, media was replaced with fresh DMEM/10% FBS. The cells were then selected by growing the cells in 10% FBS in DMEM cell media with hygromycin (200 μg/ml) as a selection reagent. The expression of the chimera clones in the cell lines was determined using CRF-BP mouse monoclonal antibody against full length CRF-BP (human origin). 50 μg of cell lysate was loaded per well on 4-20% Tris-Glycine gel from Invitrogen (Carlsbad, Calif.).

These chimeras were further tested for functionality by measuring their activity in activation and inhibition assays performed as described in the following examples.

### Example 6

### CRF-Induced Signaling through the CRF-BP_NK₁R Chimeras

The mammalian cells expressing the various isolated chimera constructs were tested for the ability of the chimera proteins to activate intracellular calcium release. The HEK-293 cells of Example 5 expressing the constructs of Example 4 were grown in 10% FBS in DMEM cell media with hygromycin (0.4%) as a selection reagent. Cells were plated out in 96-well plates (40 000 cells/well) in FBS/DMEM media. On the following day, cells were serum starved (1% FBS in DMEM; 100 μl/well) for 2 hours prior to testing.

Cells were loaded with diluted FLIPR™ dye (100 μl) for one hour prior to testing with CRF. The selected hygromycin resistant cells were plated in 96-well clear bottom black microplates at a density of approximately 40,000 cells/well, in DMEM/10% FBS media. One vial of Ca²⁺ dye (Molecular Devices) was diluted in 10 ml of 1× Washbuffer [10 mL 10× Hank's Balanced Salt Solution, 2 mL HEPES 1 M, 87 mL distilled water, 1 mL Probenecid 250 mM (71 mg dissolved in 1 ml 1N NaOH), Set pH to 7.4]. On the day of the assay, 100 μl of the diluted Ca²⁺ dye was added to each well containing the cells, for a total volume of 200 μl/well. The plates were incubated for 60 minutes at 37° C. The cells were then treated with CRF in a range of concentrations (10 pM-10 μM). This was added to individual wells in a volume of 50 μL per well.

A FlexStation (Molecular Devices) fluorometric imaging plate reader was used to measure changes in intracellular Ca²⁺. The plates were placed in the FlexStation for the assay. The machine was used in Flex mode, and the fluorescent intensity was measured from the bottom with the excitation at 485 nm and emission at 525 nm for 120 seconds at 21° C.

The results of the FLIPR experiments detecting the levels of intracellular calcium induced by activation of the chimeras with CRF are shown in FIG. 9. The chimera displayed different levels of CRF-induced (1 pM-10 μM) intracellular calcium release in the HEK-293 cells corresponding to the concentration of CRF. Results are expressed as the mean (±SEM) relative fluorescence units (RFU), calculated as agonist-induced maximum Ca²⁺ peak/cell number×1000.

This experiment demonstrates that CRF interacts with the CRF-BP_{—} NK₁R chimera, and is able to signal though the chimera construct to control intracellular calcium release. This result suggests that CRF-BP is indeed at the surface and involved in modulating NK₁R.

As with the CRF-BP_CRFR2 chimera, the CRF-BP_NK₁R assays were performed on a smaller scale in 96 well plates. This assay is likewise scalable to a 384 well format to facilitate screening of test agents that modulate CRF signaling through NK₁R in vivo.

### Example 7

### Substance P-Induced Signaling through the CRF-BP_NK₁R Chimeras

The mammalian cells expressing the various isolated chimera constructs were tested for the ability of the chimera proteins to activate intracellular calcium release by activation with Substance P, which is the endogenous ligand for NK₁R. The HEK-293 cells of Example 5 expressing the constructs of Example 4, as well as HEK 293 cells expressing the NK₁R, were grown in 10% FBS in DMEM cell media with hygromycin (0.4%) as a selection reagent. Cells were plated out in 96-well plates (40 000 cells/well) in FBS/DMEM media. On the following day, cells were serum starved (1% FBS in DMEM; 100 μl/well) for 2 hours prior to testing.

Cells were loaded with the diluted FLIPR™ dye (100 μl) for one hour prior to testing with CRF. The selected hygromycin resistant cells were plated in 96-well clear bottom black microplates at a density of approximately 40,000 cells/well, in DMEM/10% FBS media. One vial of Ca²⁺ dye (Molecular Devices) was diluted in 10 ml of 1× Washbuffer [10 mL 10× Hank's Balanced Salt Solution, 2 mL HEPES 1 M, 87 mL distilled water, 1 mL Probenecid 250 mM (71 mg dissolved in 1 ml 1N NaOH), Set pH to 7.4]. On the day of the assay, 100 μl of the diluted Ca²⁺ dye was added to each well containing the cells, for a total volume of 200 μl/well. The plates were incubated for 60 minutes at 37° C. The cells were then treated with Substance P in a range of concentrations (10 pM-10 μM). This was added to individual wells in a volume of 50 μL per well.

A FlexStation (Molecular Devices) fluorometric imaging plate reader was used to measure changes in intracellular Ca²⁺. The plates were placed in the FlexStation for the assay. The machine was used in Flex mode, and the fluorescent intensity was measured from the bottom with the excitation at 485 nm and emission at 525 nm for 120 seconds at 21° C.

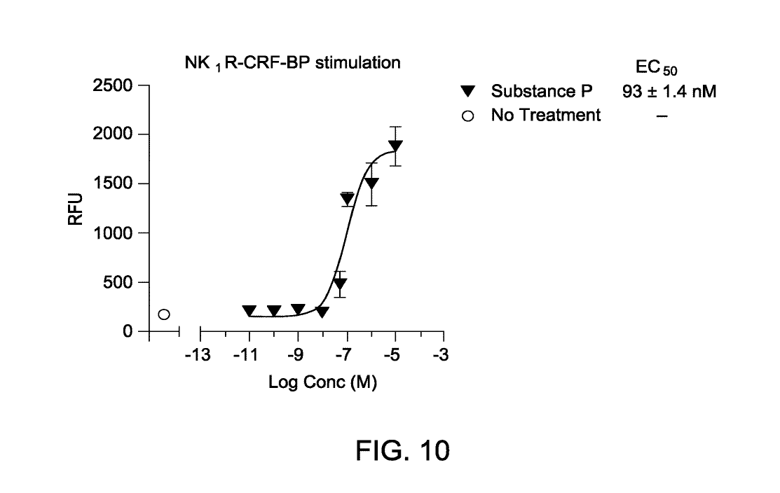

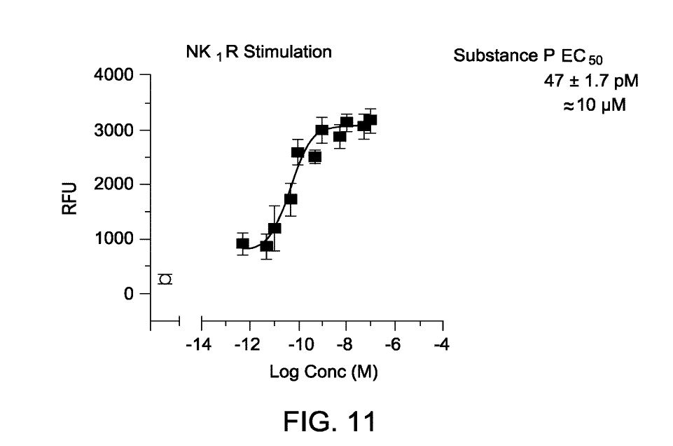

The results of the FLIPR experiments detecting the levels of intracellular calcium induced by activation of the chimeras with Substance P is shown in FIG. 10. The chimera displayed different levels of Substance P-induced (1 pM-10 μM) intracellular calcium release in the HEK-293 cells corresponding to the concentration of Substance P. Results are expressed as the mean (±SEM) relative fluorescence units (RFU), calculated as agonist-induced maximum Ca²⁺ peak/cell number×1000. The resulting calcium release seen with the chimeras is similar to (although slightly lower) than the intracellular calcium release achieved in release in HEK-293 cells expressing the full-length NK₁-R (FIG. 11). The heterodimer complex displayed different levels of Substance P (1 pM-10 μM). This experiment demonstrates that Substance P interacts with the CRF-BP_NK₁R chimera, and is able to signal though the chimera construct, presumably through direct interaction with NK₁R, to control intracellular calcium release. This result suggests that the chimera is still modulated using the endogenous ligand to the receptor portion of the chimera.

### Example 8

### Cloning and Production of a IGF-BP2_CRFR Chimera

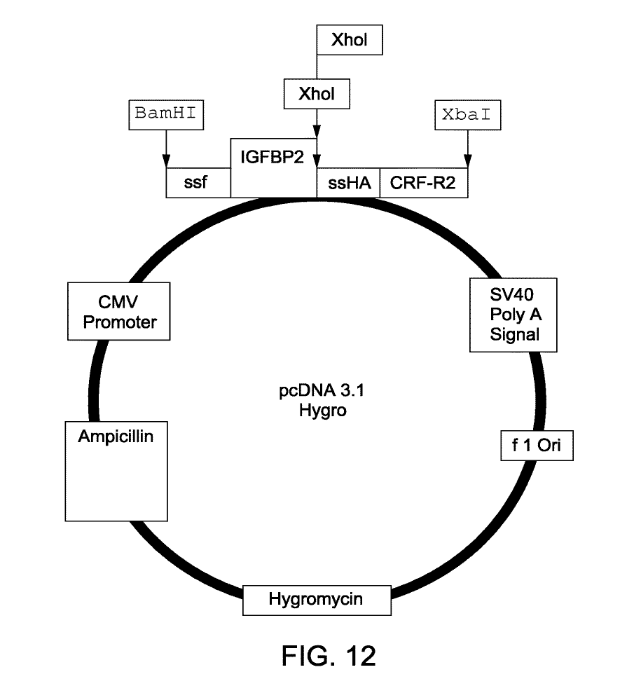

Following demonstration of the ability of chimeras comprising CRF-BP to allow signaling through the GPCRs, a chimera with another binding protein, IGF-BP2, was tested. These experiments demonstrated that the binding protein element of the GPCR chimera compositions reflects a more general approach to using different binding proteins with different GPCR classes. The map of the vector produced for the expression of the IGF-BP2_CRFR chimera is shown in FIG. 12. Construction of this plasmid is as described below.

A pcDNA3.1 Hygro vector (Invitrogen, Carlsbad, Calif.) and IGF-BP2 clone (Origene) were digested with BamHI, and XhoI (NEB, Ipswich, Mass.), and the digested DNA was run on a 1.2% agarose gel at 70 volts for 50 min and the desired fragments purified using a Qiagen (Valencia, Calif.) Gel Extraction Kit. Following elution in water, the two DNA components were ligated into a recircularized vector.

Chemically competent E. Coli cells (Invitrogen, Carlsbad, Calif.) were transformed with the vector, plated on LB plates, and allowed to incubate overnight at 37° C. The cells were then cultured in 5 ml LB, and the vector isolated via a standard plasmid preparation using a Qiagen (Ventura, Calif.) mini prep kit. The sequence of the plasmids was confirmed by restriction endonuclease digestion and sequencing.

DNA encoding the CRFR2 protein was amplified using human complementary DNA (cDNA) and the following primers: ATATCTAGATATCCTTACGACGTGCCTGA(forward) (SEQ ID NO:7) and ATATCTAGAAATTCGCCCTTGTCGACTC (reverse) (SEQ ID NO:5). The XbaI digestion site in the primers is shown in bold. 1.5 μl (approximately 150 ng) of human cDNA was used as template DNA in the PCR reaction, and 2.5 μl of each primer at a concentration of 5 μM. The DNA was amplified using 0.2 μl Highfidelity Taq at 5 u/μl in a total volume of 50 μl. PCR conditions were 30 cycles of 94° C. for 30 seconds, 59° C. for 30 seconds and 68° C. for 30 seconds, followed by a final extension at 68° C. for 5 minutes. The PCR products were then purified using PCR purification kit from Qiagen (Valencia, Calif.)

The purified CRFR2 DNA and the pcDNA 3.1 vector were both digested with XbaI (NEB, Ipswich, Mass.), and the desired fragments gel purified using a Qiagen (Valencia, Calif.) Gel Extraction Kit. The digested PCR product and vector DNA were ligated using T4 DNA ligase. The ligation reaction included 1 μl purified vector DNA, 2 μl purified CRFR2 DNA, 2 μl 10× ligase buffer, 1 μl T4 DNA ligase (NEB, Ipswich, Mass.) and 14 μl water. The ligation reaction mixture was incubated at 16° C. overnight, followed by incubation at 65° C. for 20 minutes. It was stored at 4° C. until further use as described below.

Chemically competent E. Coli cells (Invitrogen, Carlsbad, Calif.) were transformed with the ligated CRFR2 vector, plated on LB plates, and allowed to incubate overnight at 37° C. The cells were then cultured in 5 ml LB, and the vector isolated via a standard plasmid preparation. The sequence of the plasmids and the desired orientation of the insert for proper expression was confirmed by restriction endonuclease digestion and sequencing.

Human FLAG tagged IGF-BP2 DNA was then amplified using human cDNA and the following primers: ATATCTAGATATCCTTACGACGTGCCTGA(forward) (SEQ ID NO:7) and ATATCTAGAAATTCGCCCTTGTCGACTC (reverse) (SEQ ID NO: 5). The XhoI binding site is shown in bold. 1.5 μl (approximately 150 ng) of human cDNA) was used as template DNA in the PCR reaction, and 2.5 μl of each primer at a concentration of 5 μM. The DNA was amplified using 0.2 μl Highfidelity Taq at 5 u/μl in a total volume of 50 μl. PCR conditions were 30 cycles of 94° C. for 30 seconds, 59° C. for 30 seconds and 68° C. for 30 seconds, followed by a final extension at 68° C. for 5 minutes. The PCR products were then purified using PCR purification kit from Qiagen (Valencia, Calif.).

The purified FLAG-IGF-BP PCR product and the HA tagged CRFR2_pcDNA plasmid were both digested with XhoI (NEB, Ipswich, Mass.), and the desired fragments purified using a Qiagen (Valencia, Calif.) Gel Extraction Kit. The digested PCR product and vector DNA were ligated using T4 DNA ligase. The ligation reaction included 1 μl purified vector DNA, 2 μl purified CRFR2 DNA, 2 μl 10× ligase buffer, 1 μl T4 DNA ligase (NEB, Ipswich, Mass.) and 14 μl water. The ligation reaction mixture was incubated at 16° C. overnight, followed by incubation at 65° C. for 20 minutes.

The resulting plasmid contains DNA encoding FLAG tagged IGF-BP 5′ to the HA fragment. The CRFR2 coding region is in-frame with the CFR-BP, so that any protein created using this plasmid will result in a protein having the FLAG-IGF-BP component at its N-terminus, the HA components, and the CRFR2 portions in frame and at the carboxy-terminus

Chemically competent E. Coli cells (Invitrogen, Carlsbad, Calif.) were transformed with the ligated CRFR2 vector, plated on LB plates, and allowed to incubate overnight at 37° C. The cells were then cultured in 5 ml LB, and the vector isolated via a standard plasmid preparation using a Qiagen (Ventura, Calif.) mini prep kit. The sequence of the plasmids and the desire orientation for expression was confirmed by restriction endonuclease digestion and sequencing.

### Example 9

### Transfection of Human Cells with the Expression Plasmid

The plasmid containing the fusion chimera was then transfected into HEK 293 cells for expression of the protein, confirmation of the appropriate insertion into the membrane, and functionality of the chimera in mammalian cells.

One day prior to transfection, HEK 293 cells were placed in a 12 well plate with DMEM with 10% fetal bovine serum (FBS) in each of the wells. Immediately prior to transfection, each well was examined to confirm approximately 90-95% confluency of the cells in the wells. The DMEM was carefully aspirated from the wells, and replaced with fresh DMEM/10% FBS.

The final DNA plasmid preparation created in Example 8 was then diluted in 100 μl of Opti-MEM (reduced serum) and gently mixed. Lipofectamine 2000 which had been likewise diluted in 100 μl of Opti-MEM was incubated at room temperature for 5 minutes, and then combined with the diluted DNA. This was mixed gently and incubated at room temperature for 20 minutes.

Approximately 200 μl of the Lipofectamine 2000-DNA complexes were added to each well to a total volume of 1.2 ml/well. The well contents were mixed gently by rocking the plate back and forth, and incubated at 37° C. for 5 hours. After incubation media was replaced with fresh DMEM/10% FBS. The cells were then selected by growing the cells in 10% FBS in DMEM cell media with hygromycin (200 μg/ml) selection reagent. The expression of the chimera clones in the cell lines was determined using IGF-BP mouse monoclonal antibody against full length IGF-BP (human origin). 50 μg of cell lysate was loaded per well on 4-20% Tris-Glycine gel from Invitrogen (Carlsbad, Calif.).

These chimeras were further tested for functionality by measuring their activity in activation and inhibition assays performed as described in the following examples.

### Example 10

### CRF-Induced Signaling through the IGF-BP_CRFR2 Chimeras

The mammalian cells expressing the various isolated chimera constructs were tested for the ability of the chimera proteins to activate intracellular calcium release. The HEK-293 cells expressing the constructs of Example 9 were grown in 10% FBS in DMEM cell media with hygromycin (0.4%) selection reagent. The cells were plated out in 96-well plates (40 000 cells/well) in FBS/DMEM media. On the following day, cells were serum starved (1% FBS in DMEM; 100 μl/well) for 2 hours prior to testing.

The cells were loaded with diluted FLIPR™ dye (100 μl) for one hour prior to testing with CRF. The selected hygromycin resistant cells were plated in 96-well clear bottom black microplates at a density of approximately 40,000 cells/well, in DMEM/10% FBS media. One vial of Ca²⁺ dye (Molecular Devices) was diluted in 10 ml of 1× Washbuffer [10 mL 10× Hank's Balanced Salt Solution, 2 mL HEPES 1 M, 87 mL distilled water, 1 mL Probenecid 250 mM (71 mg dissolved in 1 ml 1N NaOH), Set pH to 7.4]. On the day of the assay, 100 μl of the diluted Ca²⁺ dye was added to each well containing the cells, for a total volume of 200 μl/well. The plates were incubated for 60 minutes at 37° C. The cells were then treated with CRF in a range of concentrations (10 pM-10 μM). This was added to individual wells in a volume of 50 μL per well.

A FlexStation (Molecular Devices) fluorometric imaging plate reader was used to measure changes in intracellular Ca²⁺. The plates were placed in the FlexStation for the assay. The machine was used in Flex mode, and the fluorescent intensity was measured from the bottom with the excitation at 485 nm and emission at 525 nm for 120 seconds at 21° C.

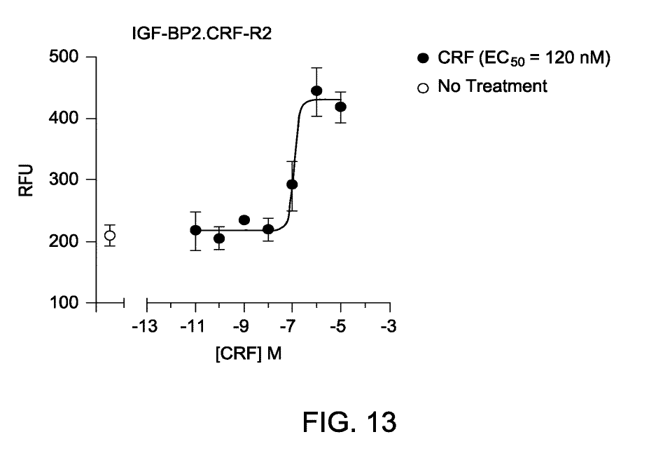

The results of the FLIPR experiments detecting the levels of intracellular calcium induced by activation of the chimeras with CRF are shown in FIG. 13. The chimera displayed different levels of CRF-induced (1 pM-10 μM) intracellular calcium release in the HEK-293 cells corresponding to the concentration of CRF. Results are expressed as the mean (±SEM) relative fluorescence units (RFU), calculated as agonist-induced maximum Ca²⁺ peak/cell number×1000.

This experiment demonstrates that CRF interacts with the IGF-BP2_CRFR chimera, and is able to signal though the chimera construct to control intracellular calcium release. This result suggests that IGF-BP2 is indeed at the surface and somehow facilitates modulation of CRFR2.

### Example 11

### Cloning and Production of a EGFR CRFR2 Chimera

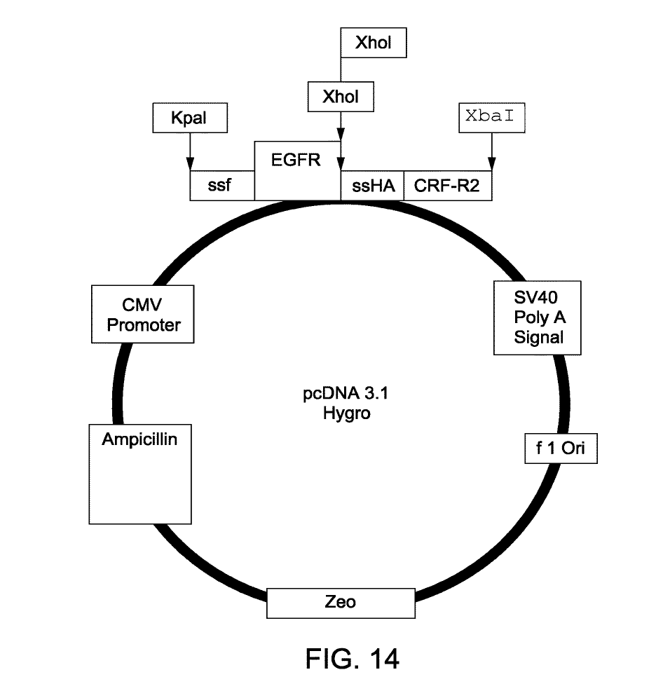

It is also known that GPCRs associate with other transmembrane receptors in the signaling of different systems. To demonstrate that the chimeras of the invention could also be produced using a transmembrane receptor and a GPCR, a human EGFR_CRFR2 chimera was produced by initial cloning of the first GPCR peptide and second signaling complex peptide into a pcDNA3.1 vector. The map of the vector produced for the expression of the EGFR_CRFR2 chimera is shown in FIG. 14. Construction of this plasmid is as described below.

### Example 12

### Transfection of Human Cells with the Expression Plasmid

The plasmid containing the fusion chimera was then transfected into HEK 293 cells for expression of the protein, confirmation of the appropriate insertion into the membrane, and functionality of the chimera in mammalian cells.

One day prior to transfection, HEK 293 cells were placed in a 12 well plate with DMEM with 10% fetal bovine serum (FBS) in each of the wells. Immediately prior to transfection, each well was examined to confirm approximately 90-95% confluency of the cells in the wells. The DMEM was carefully aspirated from the wells, and replaced with fresh DMEM/10% FBS.

The final DNA plasmid preparation created in Example 11 was then diluted in 100 μl of Opti-MEM (reduced serum) and gently mixed. Lipofectamine 2000 which had been likewise diluted in 100 μl of Opti-MEM was incubated at room temperature for 5 minutes, and then combined with the diluted DNA. This was mixed gently and incubated at room temperature for 20 minutes.

Approximately 200 μl of the Lipofectamine 2000-DNA complexes were added to each well to a total volume of 1.2 ml/well. The well contents were mixed gently by rocking the plate back and forth, and incubated at 37° C. for 5 hours. After incubation, media was replaced with fresh DMEM/10% FBS. The cells were then selected by growing the cells in 10% FBS in DMEM cell media with hygromycin (200 μg/ml) selection reagent.

The cell surface expression of the EGFR chimeras was demonstrated using immunohistochemistry. The EGFR and CRFR portions of the chimera were both detectable on the cell surface, demonstrating appropriate cell membrane insertion of the molecule (data not shown).

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims. In the claims that follow, unless the term “means” is used, none of the features or elements recited therein should be construed as means-plus-function limitations pursuant to 35 U.S.C. §112, ¶6.

What is claimed is:

1. A composition comprising:
a first peptide having an N-terminal extracellular domain from a GPCR, a transmembrane region from a GPCR, and an intracellular signaling domain from a GPCR; and
a second peptide that corresponds to a binding partner known to associate in a binding complex with a GPCR in the modulation of a biological process;
wherein the second peptide is fused to the first peptide, and wherein the expression of the fused peptides preserves GPCR signaling activity of the first peptide in a functional assay.

2. The composition of claim 1, wherein the second peptide is fused to the N-terminal extracellular domain of the first peptide.

3. The composition of claim 1, wherein the intracellular signaling domain and the transmembrane region of the first peptide correspond to the same GPCR.

4. The composition of claim 1, wherein the transmembrane region and the N-terminal extracellular domain of the first peptide correspond to the same GPCR.

5. The composition of claim 1, wherein the first peptide comprises a substantially complete amino acid sequence of a GPCR.

6. The composition of claim 5, wherein the GPCR is a Class A GPCR.

7. The composition of claim 5, wherein the GPCR is a Class B GPCR.

8. The composition of claim 5, wherein the GPCR is a Class C GPCR.

9. A research tool, comprising the composition of claim 1.

10. Use of the research tool of claim 9 in the discovery of a therapeutic agent.

11. Use of the research tool of claim 9 as a diagnostic agent.

12. A binding partner to the composition of claim 1 identified using the research tool of claim 9.

13. Use of the binding partner of claim 12 in a therapeutic setting.

14. A method for identification of a drug candidate for treatment of a biological process involving signaling through a GPCR, said method comprising:
providing a research tool composition comprising:
a first peptide having an N-terminal extracellular domain from a GPCR, a transmembrane region from a GPCR, and an intracellular signaling domain from a GPCR; and
a second peptide that corresponds to a binding partner known to associate in a binding complex with a GPCR in the modulation of a biological process;
wherein the second peptide is fused to the first peptide, and wherein the expression of the fused peptides preserves GPCR signaling activity of the first peptide in a functional assay;
testing one or more binding partners for modulation of functional activity of the research tool composition, and
isolating the binding partners that display the desired change in functional activity of the research tool composition;
wherein the binding partners that display the desired change in functional activity of the research tool composition are drug candidates for the biological process involving signaling through the GPCR.

15. The method of claim 14, wherein second peptide is fused to the N-terminal extracellular domain of the first peptide.

16. The method of claim 14, wherein the intracellular signaling domain and the transmembrane region of the first peptide of the research tool composition correspond to the same GPCR.

17. The method of claim 14, wherein the transmembrane region and the N-terminal extracellular domain of the first peptide of the research tool composition correspond to the same GPCR.

18. The method of claim 14, wherein the first peptide of the research tool composition comprises a substantially complete amino acid sequence of a GPCR.


SEQUENCE LISTING (9 sequences)

SEQ ID NO: 1 - protein, 88 aa, Homo sapiens
AGCEGIGDFVELLGGTGLDPSKMTPLADLCYPFHGPAQMKVGCDNTVVRMVSSGKHVNRVTFEYRQLEPYELENPNGNSIGEFCLSGL

SEQ ID NO: 2 - DNA, 29 bp, homo sapiens
ataggatccggcaggttgcgagggaatag

SEQ ID NO: 3 - DNA, 27 bp, homo sapiens
atactcgagtagaaggcacagtcgagg

SEQ ID NO: 4 - DNA, 29 bp, Homo sapiens
atactcgagtatccttacgacgtgcctga

SEQ ID NO: 5 - DNA, 28 bp, homo sapiens
atatctagaaattcgcccttgtcgactc

SEQ ID NO: 6 - protein, 28 aa, homo sapiens
LVSAGVLLVALLPCPPCRALLSRGPVPG

SEQ ID NO: 7 - DNA, 29 bp, homo sapiens
atatctagatatccttacgacgtgcctga

SEQ ID NO: 8 - DNA, 25 bp, homo sapiens
ataaagcttaccatgaagacgatca

SEQ ID NO: 9 - DNA, 31 bp, homo sapiens
ataaagcttagacaaacagaattccccgata